SEQUENCE LISTING (15 sequences)

SEQ ID NO: 1 - DNA, 10 bp, synthetic construct
  source 1..10: mol_type = other DNA
gactagtctg

SEQ ID NO: 2 - DNA, 14 bp, Homo sapiens
  source 1..14: mol_type = genomic DNA
accacactgcaaac

SEQ ID NO: 3 - DNA, 15 bp, synthetic construct
  source 1..15: mol_type = other DNA
accacacttgcaaac

SEQ ID NO: 4 - DNA, 13 bp, synthetic construct
  source 1..13: mol_type = other DNA
accaacaatagcc

SEQ ID NO: 5 - DNA, 12 bp, synthetic construct
  source 1..12: mol_type = other DNA
accacaatagcc

SEQ ID NO: 6 - DNA, 20 bp, synthetic construct
  source 1..20: mol_type = other DNA
cactgcaaacaatagccacc

SEQ ID NO: 7 - DNA, 46 bp, Homo sapiens
  source 1..46: mol_type = genomic DNA
gatcaggaacctctccaccacactgcaaacaatagccaccaaatta

SEQ ID NO: 8 - DNA, 23 bp, Homo sapiens
  source 1..23: mol_type = genomic DNA
ctccaccacactgcaaacaatag

SEQ ID NO: 9 - DNA, 18 bp, Homo sapiens
  source 1..18: mol_type = genomic DNA
aagcaatcgactccaact

SEQ ID NO: 10 - DNA, 18 bp, synthetic construct
  source 1..18: mol_type = other DNA
aacaactggagcctcgca

SEQ ID NO: 11 - DNA, 18 bp, synthetic construct
  source 1..18: mol_type = other DNA
aacaatgcacgttaatgg

SEQ ID NO: 12 - DNA, 18 bp, synthetic construct
  source 1..18: mol_type = other DNA
cacatatcaacctagcac

SEQ ID NO: 13 - DNA, 18 bp, synthetic construct
  source 1..18: mol_type = other DNA
aggaaggtgcaccaagat

SEQ ID NO: 14 - DNA, 22 bp, Homo sapiens
  source 1..22: mol_type = genomic DNA
ctccaccacactgcaaacaata

SEQ ID NO: 15 - DNA, 10 bp, synthetic construct
  source 1..10: mol_type = other DNA
cacactgcaa

(19) United States
(12) Patent Application Publication
(10) Pub. No.: US 2025/0140342 A1
(43) Pub. Date: May 1, 2025

(54) GENOTYPING CELLS USING SINGLE-CELL DNA SEQUENCING DATA

(71) Applicant: VOR BIOPHARMA INC., Cambridge, MA (US)

(72) Inventors: Matthew HaoJon Ung, Cambridge, MA (US); Ruijia Wang, Cambridge, MA (US); Gary Huanying Ge, Cambridge, MA (US); Tirtha Chakraborty, Cambridge, MA (US)

(73) Assignee: VOR BIOPHARMA INC., Cambridge, MA (US)

(21) Appl. No.: 18/920,753

(22) Filed: Oct. 18, 2024

Related U.S. Application Data
(60) Provisional application No. 63/592,539, filed on Oct. 23, 2023.

(51) Int. Cl. G16B 20/20; C12Q 1/6869; G16B 30/20

(52) U.S. Cl. CPC G16B 20/20; C12Q 1/6869; G16B 30/20

(57) ABSTRACT

Some aspects provide for techniques for genotyping cells in a biological sample. In some embodiments, the techniques include: obtaining single cell DNA sequence (scDNA-seq) data for a plurality of droplets; and genotyping the cells using the scDNA-seq data, the genotyping comprising determining for each droplet, a genotype for a locus of a respective genome of at least one cell associated with the droplet, the determining comprising: identifying, from among the plurality of droplets, a first set of droplets associated with cells that are homozygous at the locus; identifying, from among the plurality of droplets not in the first set of droplets, a second set of droplets associated with more than two alleles at the locus; and identifying, from among the plurality of droplets not in the first or second sets of droplets, a third set of droplets associated with cells that are heterozygous at the locus.

### CROSS-REFERENCE TO RELATED APPLICATIONS

This application claims the benefit under 35 U.S.C. § 119(e) of the filing date of U.S. Provisional Application No. 63/592,539, filed Oct. 23, 2023, and entitled “GENOTYPING CELLS USING SINGLE-CELL DNA SEQUENCING DATA,” the entire contents of which are incorporated by reference herein.

### SEQUENCE LISTING

The instant application contains a Sequence Listing which has been submitted electronically in XML format and is hereby incorporated by reference in its entirety. Said XML copy, created on Nov. 27, 2024, is named V029170047US01-SEQ-DGR.xml and is 14,207 bytes in size.

### BACKGROUND

Gene editing includes techniques for modifying DNA in the genome of an organism. It can involve adding, removing, or altering genetic material at particular locations in the genome. There are multiple approaches to gene editing including, for example, CRISPR-Cas9.

Droplet-based targeted single-cell DNA sequencing (scDNA-seq) can be used to genotype loci across thousands of cells. It can be applied to genotype cells after gene editing.

### SUMMARY

Some aspects provide for a method for genotyping a plurality of cells in a biological sample using single-cell DNA sequencing (scDNA-seq) data obtained for a plurality of droplets, each of the plurality of droplets being associated with at least one cell of the plurality of cells, the method comprising: using at least one computer hardware processor to perform: obtaining the scDNA-seq data for the plurality of droplets, the scDNA-seq data having been previously obtained by sequencing the plurality of cells using scDNA-seq, wherein the scDNA-seq data comprises values indicative of frequencies of one or more alleles at a locus, the values including, for each particular droplet of the plurality of droplets, one or more values indicative of respective frequencies of the one or more alleles at the locus of a genome of at least one cell associated with the particular droplet; and genotyping the plurality of cells using the scDNA-seq data to obtain a respective plurality of cell genotypes, the genotyping comprising determining, using the scDNA-seq data, for each particular droplet of the plurality of droplets, a genotype for the locus of the respective genome of the at least one cell associated with the particular droplet, the determining comprising: identifying, using the scDNA-seq data and from among the plurality of droplets, a first set of droplets associated with cells that are homozygous at the locus; identifying, using the scDNA-seq data and from among the plurality of droplets not in the first set of droplets, a second set of droplets associated with more than two alleles at the locus; and identifying, using the scDNA-seq data and from among the plurality of droplets not in the first or second sets of droplets, a third set of droplets associated with cells that are heterozygous at the locus.

Some aspects provide for a system, comprising: at least one computer hardware processor; and at least one non-transitory computer-readable storage medium storing processor-executable instructions that, when executed by the at least one computer hardware processor, cause the at least one computer hardware processor to perform a method for genotyping a plurality of cells in a biological sample using single-cell DNA sequencing (scDNA-seq) data obtained for a plurality of droplets, each of the plurality of droplets being associated with at least one cell of the plurality of cells, the method comprising: obtaining the scDNA-seq data for the plurality of droplets, the scDNA-seq data having been previously obtained by sequencing the plurality of cells using scDNA-seq, wherein the scDNA-seq data comprises values indicative of frequencies of one or more alleles at a locus, the values including, for each particular droplet of the plurality of droplets, one or more values indicative of respective frequencies of the one or more alleles at the locus of a genome of at least one cell associated with the particular droplet; and genotyping the plurality of cells using the scDNA-seq data to obtain a respective plurality of cell genotypes, the genotyping comprising determining, using the scDNA-seq data, for each particular droplet of the plurality of droplets, a genotype for the locus of the respective genome of the at least one cell associated with the particular droplet, the determining comprising: identifying, using the scDNA-seq data and from among the plurality of droplets, a first set of droplets associated with cells that are homozygous at the locus; identifying, using the scDNA-seq data and from among the plurality of droplets not in the first set of droplets, a second set of droplets associated with more than two alleles at the locus; and identifying, using the scDNA-seq data and from among the plurality of droplets not in the first or second sets of droplets, a third set of droplets associated with cells that are heterozygous at the locus.

Some aspects provide for at least one non-transitory computer-readable storage medium storing processor-executable instructions that, when executed by at least one computer hardware processor, cause the at least one computer hardware processor to perform a method for genotyping a plurality of cells in a biological sample using single-cell DNA sequencing (scDNA-seq) data obtained for a plurality of droplets, each of the plurality of droplets being associated with at least one cell of the plurality of cells, the method comprising: obtaining the scDNA-seq data for the plurality of droplets, the scDNA-seq data having been previously obtained by sequencing the plurality of cells using scDNA-seq, wherein the scDNA-seq data comprises values indicative of frequencies of one or more alleles at a locus, the values including, for each particular droplet of the plurality of droplets, one or more values indicative of respective frequencies of the one or more alleles at the locus of a genome of at least one cell associated with the particular droplet; and genotyping the plurality of cells using the scDNA-seq data to obtain a respective plurality of cell genotypes, the genotyping comprising determining, using the scDNA-seq data, for each particular droplet of the plurality of droplets, a genotype for the locus of the respective genome of the at least one cell associated with the particular droplet, the determining comprising: identifying, using the scDNA-seq data and from among the plurality of droplets, a first set of droplets associated with cells that are homozygous at the locus; identifying, using the scDNA-seq data and from among the plurality of droplets not in the first set of droplets, a second set of droplets associated with more than two alleles at the locus; and identifying, using the scDNA-seq data and from among the plurality of droplets not in the first or second sets of droplets, a third set of droplets associated with cells that are heterozygous at the locus.

Embodiments of any of the above aspects may have one or more of the following features.

In some embodiments, the biological sample was previously processed using CRISPR-Cas9 gene editing.

Some embodiments further comprise: processing the biological sample using CRISPR-Cas9 gene editing.

Some embodiments further comprise: regulating treatment of a second biological sample based on the plurality of cell genotypes.

In some embodiments, regulating the treatment of the second biological sample comprises outputting, based on the plurality of cell genotypes, a recommendation for modifying a manner in which one or more materials are added to the second biological sample.

In some embodiments, regulating the treatment of the second biological sample comprises modifying a manner in which one or more materials are added to second biological sample.

Some embodiments further comprise regulating treatment of the biological sample based on the plurality of cell genotypes.

In some embodiments, regulating the treatment of the biological sample comprises outputting, based on the plurality of cell genotypes, a recommendation for expanding cells in the biological sample.

In some embodiments, regulating the treatment of the biological sample comprises expanding cells in the biological sample.

In some embodiments, identifying the first set of droplets comprises: clustering the plurality of droplets into a first set of one or more droplet clusters; and identifying a particular droplet cluster of the first set of one or more droplet clusters as the first set of droplets. In some embodiments, clustering the plurality of droplets into the first set of one or more droplet clusters comprises clustering the plurality of droplets based on dominant allele frequencies for the plurality of droplets, wherein the dominant allele frequencies are specified by the scDNA-seq data.

In some embodiments, clustering the plurality of droplets comprises: fitting a first Gaussian mixture model (GMM) to the dominant allele frequencies; and using the fitted first GMM to obtain the first set of one or more droplet clusters.

In some embodiments, identifying the second set of droplets comprises: clustering the plurality of droplets not in the first set of droplets into a second set of one or more droplet clusters; and identifying a particular droplet cluster of the second set of one or more droplet clusters as the second set of droplets. In some embodiments, clustering the plurality of droplets not in the first set of droplets comprises clustering the plurality of droplets not in the first set of droplets based on a respective plurality of ploidy scores for the plurality of droplets not in the first set of droplets.

Some embodiments further comprise determining the respective plurality of ploidy scores for the plurality of droplets not in the first set of droplets, the determining comprising determining the respective plurality of ploidy scores based on (a) minor allele counts for the plurality of droplets and (b) allele counts for a third most common allele for the plurality of droplets, wherein the minor allele counts and the allele counts for the third most common allele are specified by the scDNA-seq data.

In some embodiments, clustering the plurality of droplets not in the first set of droplets comprises: fitting a second GMM to the ploidy scores; and using the fitted second GMM to obtain the second set of one or more droplet clusters.

In some embodiments, identifying the third set of droplets comprises: clustering the plurality of droplets not in the first or second sets of droplets into a third set of one or more droplet clusters; and identifying a particular droplet cluster of the third set of one or more droplets clusters as the third set of droplets. In some embodiments, clustering the plurality of droplets not in the first or second sets of droplets comprises clustering the plurality of droplets not in the first or second sets of droplets based on principal components of allele frequencies for the plurality of droplets not in the first or second sets of droplets, wherein the allele frequencies are specified by the scDNA-seq data.

Some embodiments further comprise performing dimensionality reduction on the allele frequencies to obtain the principal components of the allele frequencies.

In some embodiments, clustering the plurality of droplets not in the first or second sets of droplets based on the principal components of the allele frequencies comprises: fitting a third GMM to the principal components of the allele frequencies; and using the fitted third GMM to obtain the third set of one or more droplet clusters.

In some embodiments, droplets not in the first, second, or third sets of droplets are each associated with multiple cells of the plurality of cells.

Some embodiments further comprise sequencing the biological sample using scDNA-seq to obtain the scDNA-seq data.

### BRIEF DESCRIPTION OF THE DRAWINGS

The accompanying drawings are not intended to be drawn to scale. For purposes of clarity, the drawings are illustrative only and are not required for enablement of the disclosure. Not every component may be labeled in every drawing. In the drawings:

FIG. 1 is a diagram of an illustrative technique for genotyping cells using single-cell DNA sequencing (scDNA-seq) data, according to some embodiments of the technology described herein.

FIG. 2A shows a graphical overview of an example procedure for generating an artificial mix of gene edited cells, according to some embodiments of the technology described herein. Figure discloses SEQ ID NO: 1.

FIG. 2B shows an example electropherogram from Sanger sequencing of cells with homozygous and heterozygous editing at a locus, according to some embodiments of the technology described herein. Figure discloses SEQ ID NOS 1-5, respectively, in order of appearance.

FIG. 2C shows bar plots indicating examples of the total number of scDNA-seq reads mapping to four possible alleles across three artificial mixes of gene-edited cells, according to some embodiments of the technology described herein.

FIG. 2D shows example read alignment profiles of droplets from an artificial mix of gene-edited cells, according to some embodiments of the technology described herein. Figure discloses SEQ ID NOS 6-7, respectively, in order of appearance.

FIG. 3A shows an example illustration of possible artifacts in scDNA-seq data and their resulting allele frequency readouts, according to some embodiments of the technology described herein.

FIG. 3B shows example read alignment profiles of droplets selected from artificial mixes of cells that display different allele combinations at a locus, according to some embodiments of the technology described herein. Figure discloses SEQ ID NO: 8.

FIG. 3C shows an example table of allele read counts for the selected droplets from the artificial mixes of cells, according to some embodiments of the technology described herein. Figure discloses SEQ ID NOS 9-13, respectively, in order of appearance.

FIG. 3D shows an example 3D scatterplot showing frequencies of each droplet's top three most frequent alleles at the locus across all artificial mixes of cells, according to some embodiments of the technology described herein.

FIG. 3E shows an example scatterplot showing a relationship between read depth and dominant allele frequency of droplets from a sample with 100% compound heterozygous edited cells, according to some embodiments of the technology described herein.

FIG. 3F shows example read alignment profiles of four droplets with rare allele combinations, according to some embodiments of the technology described herein. Figure discloses SEQ ID NOS 14-15, respectively, in order of appearance.

FIG. 4A shows an example diagram of techniques for genotyping cells using scDNA-seq data, according to some embodiments of the technology described herein.

FIG. 4B shows example histograms showing frequency of the dominant allele in cells that were genotyped according to embodiments of the technology described herein.

FIG. 4C shows example histograms of log 2 noise allele ratios for droplets with more than two detected alleles at a locus, according to some embodiments of the technology described herein.

FIG. 4D shows an example correlogram showing allele correlation structure revealing heterozygous alleles with high co-occurrence, according to some embodiments of the technology described herein.

FIG. 4E shows example scatterplots with density contours showing putative heterozygous cells in low dimensional space with distinct clusters corresponding to droplets associated with cells that are heterozygous at a locus and droplets associated with multiple cells, according to some embodiments of the technology described herein.

FIG. 4F shows example heatmaps showing scaled log frequencies of the four alleles at a locus across droplets, according to some embodiments of the technology described herein.

FIG. 5A shows a table of droplet counts in each genotype category across different artificial mixes of gene-edited cells, according to some embodiments of the technology described herein.

FIG. 5B shows example bar plots comparing sample composition estimated for different artificial mixes of gene-edited cells, according to some embodiments of the technology described herein.

FIG. 6A shows that sample composition, estimated according to embodiments of the technology described herein, aligns with reported sample compositions.

FIG. 6B shows an example scatterplot showing a correlation between a percent of unedited cells, estimated according to embodiments of the technology described herein, and the reported percent of unedited cells.

FIG. 6C shows example tables showing overlap between droplets identified, according to embodiments of the technology described herein, as being associated with more than one cell and those with editing at more than one locus in a gene-edited sample with a single locus targeted for gene editing.

FIG. 6D shows an example table showing overlap between droplets identified, according to embodiments of the technology described herein, as being associated with more than one cell and those with simultaneous edits across six loci in the gene-edited sample.

FIG. 6E shows an example upset plot showing a number of cells with different combinations of homozygous gene edits, according to some embodiments of the technology described herein.

FIG. 7A shows example histograms showing frequency of the dominant allele in cells in different artificial mixes of cells, according to some embodiments of the technology described herein.

FIG. 7B shows example histograms showing frequency of the dominant allele in cells that were genotyped according to some embodiments of the technology described herein.

FIG. 7C shows example histograms showing frequency of the dominant allele in cells in different artificial mixes of cells, according to some embodiments of the technology described herein.

FIG. 8A shows an example of relationships between dominant allele frequency of a droplet and classification probability, according to some embodiments of the technology described herein.

FIG. 8B shows an example of relationships between the log 2 noise allele ratio of a droplet and classification probability, according to some embodiments of the technology described herein.

FIG. 8C shows an example table showing that genotyping cells in samples with a single gene-editing targets, according to embodiments of the technology described herein, can be used to identify droplets associated with multiple cells.

FIG. 9 shows example heatmaps showing scaled log frequencies of four alleles at a locus across droplets associated with different artificial mixes of cells, according to some embodiments of the technology described herein.

FIG. 10A shows a graphical overview of an example procedure for generating an artificial mix of gene edited cells, according to some embodiments of the technology described herein.

FIG. 10B shows an example overview of techniques used to analyze scDNA-seq data obtained for cells in a biological sample, according to some embodiments of the technology described herein. Figure discloses SEQ ID NO: 1.

FIG. 11A shows an example table showing allele counts for six distinct droplet types observed in an artificial mix of cells, according to some embodiments of the technology described herein. Figure discloses SEQ ID NOS 9-13, respectively, in order of appearance.

FIG. 11B an example illustration of possible artifacts in scDNA-seq data and their resulting allele frequency readouts, according to some embodiments of the technology described herein.

FIG. 11C shows an example diagram of techniques for genotyping cells using scDNA-seq data, according to some embodiments of the technology described herein.

FIG. 11D shows example distributions of the dominant allele in cells that were genotyped according to some embodiments of the technology described herein.

FIG. 11E shows an example correlogram showing allele correlation structure revealing heterozygous alleles with high co-occurrence, according to some embodiments of the technology described herein.

FIG. 11F shows example scatterplots showing droplets identified, according to embodiments of the technology described herein, as being associated with more than one cell.

FIG. 12A shows example heatmaps showing scaled log frequencies of four alleles at a locus across droplets associated with different artificial mixes of cells, according to some embodiments of the technology described herein.

FIG. 12B shows example bar plots comparing estimated and true artificial cell mix genotype compositions after removing droplets associated with multiple cells, according to some embodiments of the technology described herein.

FIG. 12C shows simulating heterozygous composition of an artificial cell mix by in silico spike-in, according to some embodiments of the technology described herein.

FIG. 13 is a schematic diagram of an illustrative computing device with which aspects described herein may be implemented.

### DETAILED DESCRIPTION

The inventors have developed techniques for genotyping cells in a biological sample using single-cell DNA sequencing (scDNA-seq) data obtained for droplets associated with the cells. In some embodiments, the techniques for genotyping cells include determining, for each droplet, a genotype for a locus of the genome of the cell(s) associated with the droplet¹. In some embodiments, determining the genotypes includes (a) identifying, using the scDNA-seq data, a first set of droplets associated with cells that are homozygous at the locus, (b) identifying, using the scDNA-seq data, a second set of droplets for which the scDNA-seq data indicates more than two alleles at the locus, and (c) identifying, using the scDNA-seq data, a third set of droplets associated with cells that are heterozygous at the locus. In some embodiments, the determined cell genotypes may be used to regulate treatment of the biological sample or regulate treatment of another (e.g., a subsequent) biological sample. ¹ A droplet may include one cell or multiple cells. Cell(s) associated with a droplet are the cell or cells in the droplet.

Genome editing technologies, such as CRISPR-Cas-based systems (e.g., CRISPR-Cas9), enable precise modification of genes, offering opportunities to decipher gene function, study disease mechanisms, and develop therapeutic strategies. Such CRISPR-Cas systems comprise the use of a RNA-guided nuclease, e.g., a CRISPR/Cas nuclease such as Cas9, to introduce targeted single- or double-stranded DNA breaks in the genome of a cell, which trigger cellular repair mechanisms, such as, for example, nonhomologous end joining (NHEJ) or microhomology-mediated end joining (MMEJ, also sometimes referred to as “alternative NHEJ” or “alt-NHEJ”). See, e.g., Yeh et al. Nat. Cell. Biol. (2019) 21:1468-1478; e.g., Hsu et al. Cell (2014) 157:1262-1278; Jasin et al. DNA Repair (2016) 44:6-16; Sfeir et al. Trends Biochem. Sci. (2015) 40:701-714, each of which is incorporated by reference herein in its entirety.

Yet another exemplary suitable genome editing technology includes “prime editing,” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157, which is incorporated by reference herein in its entirety.

Identifying allelism is important for understanding the consequences of gene editing. Accurate allelic profiling can confirm the success of intended genetic alterations, as well as identify potential off-target effects and unintended phenotypic consequences. This knowledge is important for ensuring safety and efficacy of gene-editing approaches, particularly when applied in the context of gene therapy, regenerative medicine, and precision agriculture.

Conventional techniques for assessing allelism involve isolating and expanding single cell clones from the edited sample. Clones are then individually selected and characterized by DNA sequencing or PCR to validate the presence of the intended edit. These techniques are cumbersome, time-consuming, and low throughput.

Microfluidic-based targeted scDNA-seq has the capability to barcode and sequence tens of thousands of individual cells within a sample in a relatively brief period of time, offering a more comprehensive depiction of editing outcomes. Given its ability to provide a readout at single-cell resolution, scDNA-seq may be used to profile cells with intricate genotypes across multiple loci, thereby unveiling heterogeneity of editing consequences.

There are several challenges associated with using scDNA-seq to genotype cells. First, due to its high throughput capabilities, it is challenging to rapidly and accurately evaluate allelism in single cells. For example, scDNA-seq may be used to process a biological sample having thousands of cells, which in turn results in sequencing data for each of the thousands of cells. Processing this sequence data rapidly to determine genotypes for each individual cell is computationally burdensome due to the sheer volume of data.

Second, technical artifacts in the scDNA-seq data decrease genotyping accuracy. Such technical artifacts may include, for example, low coverage at a locus, PCR amplification imbalance, sequencing error, and multiplets (e.g., doublets), which refer to droplets the encapsulate more than one cell. For example, when a cell is heterozygous at a locus, but there is low coverage of one of one of the alleles, the locus may be inaccurately genotyped as homozygous. Similarly, when a cell is heterozygous at a locus, but there is PCR amplification imbalance (e.g., greater amplification of one allele relative to the other), the locus may be inaccurately genotyped as homozygous at the locus. When multiple cells are included in a droplet, forming a multiplet, the different genotypes of the two cells may be reflected in the scDNA-seq data. For example, if, among the cells, more than two alleles are observed at the locus, then the scDNA-seq data may improperly indicate that the droplet encapsulates a heterozygous cell with more than two alleles (i.e., ploidy >2). Additionally, or alternatively, if the multiple cells are encapsulated in a droplet, the scDNA-seq data may improperly indicate that the droplet encapsulates a heterozygous cell with a rare combination of alleles at the locus. For example, this may occur when there are homozygous cells of two different allele types. Additionally, or alternatively, this may occur when there is a combination of (a) a homozygous cell and (b) low coverage or amplification bias of a different allele of another cell.

Accordingly, the inventors have developed techniques that address the above-described challenges associated with the conventional techniques for genotyping cells in a biological sample using scDNA-seq data. For example, the scDNA-seq data may include values indicative of frequencies of one or more alleles at a locus. In some embodiments, the techniques include: obtaining scDNA-seq data for a plurality of droplets, each of which is associated with at least one cell of a plurality of cells in a biological sample, and genotyping the plurality of cells using the scDNA-seq data. In some embodiments, genotyping the plurality of cells using the scDNA-seq data includes: (a) identifying, using the scDNA-seq data and from among the plurality of droplets, a first set of droplets associated with cells that are homozygous at the locus, (b) identifying, using the scDNA-seq data and from among the plurality of droplets not in the first set of droplets, a second set of droplets for which the scDNA-seq data indicates more than two alleles at the locus; and (c) identifying, using the scDNA-seq data and from among the plurality of droplets not in the first or second sets of droplets, a third set of droplets associated with cells that are heterozygous at the locus. In some embodiments, the scDNA-seq data.

The techniques developed by the inventors are an improvement over conventional techniques for genotyping cells using scDNA-seq because they accurately and efficiently distinguish among droplets associated with a single heterozygous or homozygous cell and droplets that have been affected by technical artifacts. For example, identifying the first set of droplets associated with cells that are homozygous at the locus enables accurate genotyping of homozygous cells because it involves distinguishing droplets associated with true homozygous cells from droplets affected by technical artifacts and droplets associated with heterozygous cells. Additionally, identifying the second and third sets of droplets enable the accurate genotyping of heterozygous cells because it involves distinguishing droplets associated with true heterozygous cells from droplets affected by technical artifacts. Following below are descriptions of various concepts related to, and embodiments of techniques for genotyping cells using scDNA-seq data. It should be appreciated that various aspects described herein may be implemented in any of numerous ways, as techniques are not limited to any particular manner of implementation. Examples of details of implementations are provided herein solely for illustrative purposes. Furthermore, the techniques disclosed herein may be used individually or in any suitable combination, as aspects of the technology described herein are not limited to the use of any particular technique or combination of techniques.

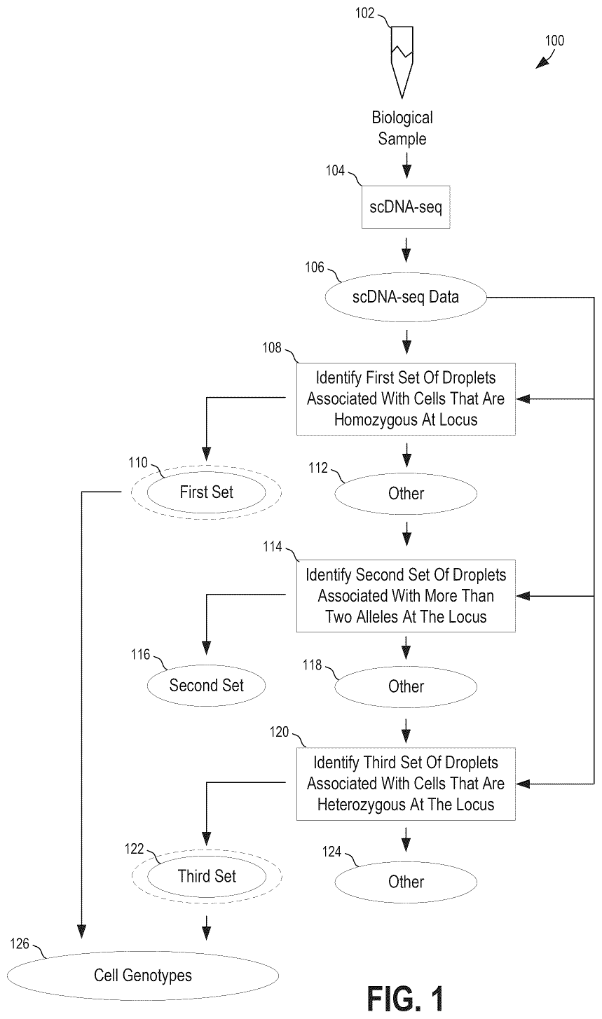

FIG. 1 is a diagram of an illustrative technique 100 for genotyping cells using single-cell DNA sequencing (scDNA-seq) data, according to some embodiments of the technology described herein. Technique 100 includes obtaining single-cell DNA sequencing (scDNA-seq) data 106 from a biological sample 102 and using the scDNA-seq data 106 to genotype cells in the biological sample 102 to obtain cell genotypes 126. In some embodiments, the scDNA-seq data 106 includes data for a plurality of droplets, and each of the droplets is associated with at least one (e.g., one, some, or all) of the cells in the biological sample 102. In some embodiments, to obtain the cell genotypes 126, technique 100 includes determining, for each droplet, a genotype for a locus of the genome(s) of the cell(s) associated with the droplet. In some embodiments, this may include, (a) at act 108, identifying, using the scDNA-seq data 106, a first set of droplets 110 associated with cells that are homozygous at the locus, (b) at act 114, identifying, using the scDNA-seq data 106, a second set of droplets 116 associated with more than two alleles at the locus, and (c) at act 120, identifying, using the scDNA-seq data 106, a third set of droplets 122 associated with cells that are heterozygous at the locus.

As shown in FIG. 1, scDNA-seq data 106 is obtained by processing biological sample 102. Biological sample 102 includes a plurality cells. In some embodiments, at least some of the cells in the biological sample 102 are gene edited. For example, the biological sample may have been previously processed using one or more gene-editing techniques. Alternatively, technique 100 may (optionally) include processing the biological sample 102 using one or more gene-editing techniques. The gene-editing technique(s) may include any suitable gene-editing technique(s), as aspects of the technology described herein are not limited in this respect. For example, cells in the biological sample 102 may be gene edited using CRISPR-Cas9 editing. An example of using a CRISPR-Cas9 system to introduce specific homozygous and heterozygous mutations is described by Paquet, D. et al., (Efficient introduction of specific homozygous and heterozygous mutations using CRISPR/Cas9. Nature 533, 125-129 (2016)), which is incorporated by reference herein in its entirety.

In some embodiments, processing biological sample 102 to obtain the scDNA-seq data 106 includes sequencing cells in the biological sample 102 using scDNA-seq. In some embodiments, sequencing cells in the biological sample 102 using scDNA-seq involves using a microfluidic droplet-based system. A microfluidic droplet-based system involves encapsulating single cells in droplets for DNA capture and amplification. Examples of sequencing cells using scDNA-seq are described by Zilionis, R. et al., (Single-cell barcoding and sequencing using droplet microfluidics. Nat Protoc. 2017 January; 12 (1): 44-73), which is incorporated by reference herein in its entirety.

In some embodiments, the output of sequencing the cells using scDNA-seq 104 includes sequence reads for at least some of the droplets used to encapsulate the cells during sequencing. In some embodiments, the sequence reads are associated with unique barcodes used to identify the droplet for which they were obtained. In some embodiments, the sequence reads are output in a file of any suitable format such as, for example, FASTQ format.

In some embodiments, the sequencing output is processed to obtain scDNA-seq data 106. In some embodiments, the scDNA-seq data 106 includes, for a droplet, values that are indicative of frequencies of different alleles at one or more loci of the cell(s) encapsulated in the droplet. For example, the scDNA-seq data may include allele read counts for each droplet. Additionally, or alternatively, the scDNA-seq data may include allele frequencies for each droplet. Allele frequencies may be determined, for example, by dividing the number of counts of an allele by the total number across all alleles per locus per droplet. In some embodiments, the scDNA-seq data may additionally, or alternatively, include information about different alleles such as, for example, an editing status of the allele, the DNA sequence, an indel profile, or any other suitable information.

In some embodiments, the sequencing output (e.g., the sequence reads) may be processed using any suitable techniques to obtain scDNA-seq data 106. In some embodiments, the processing includes aligning the sequence reads a reference genome. The reference genome may include any suitable reference genome (e.g., GRCh38.p14), as aspects of the technology described herein are not limited in this respect. Additionally, or alternatively, the processing includes barcode deconvolution for assigning the reads to cell barcodes. In some embodiments, the sequence alignment and/or barcode deconvolution may be performed using software. For example, Mission Bio's Tapestri Pipeline software may be used to perform sequence alignment and barcode deconvolution.

In some embodiments, the alignment and barcode information may be processed to obtain the scDNA-seq data. For example, in some embodiments, the alignment and barcode information may be processed using any suitable software configured to determine values indicative of allele counts and/or allele frequencies for each droplet. Such software may include, for example, CRISPResso2. CRISPResso2 is described by Clement, K., et al., (CRISPResso2 provides accurate and rapid genome editing sequence analysis. Nat Biotechnol 37, 224-226 (2019)), which is incorporated by reference herein in its entirety.

In some embodiments, software on a computing device may be configured to process at least some of the scDNA-seq data 106 to determine the cell genotypes 126. In some embodiments, this may include using the software to perform one or more of acts: 108, 114, and 120.

At act 108, a first set of droplets 110 is identified from among a plurality of droplets for which scDNA-seq data 106 was obtained. In some embodiments, the first set of droplets includes droplets associated with cells that are homozygous at a particular locus.

In some embodiments, identifying the first set of droplets 110 includes clustering the plurality of droplets into one or more droplet clusters and identifying one of the droplet clusters as the first set of droplets. In some embodiments, the clustering is performed based on dominant allele frequencies for the plurality of droplets. The dominant allele frequency may include, for example, the frequency of the most frequently occurring allele at the particular locus of a cell associated with the droplet. In some embodiments, the dominant allele frequency for a droplet may be determined using allele counts or allele frequencies indicated by the scDNA-seq data.

In some embodiments, the clustering is performed using any suitable clustering technique, as aspects of the technology described herein are not limited in this respect. For example, in some embodiments, the clustering may be performed by fitting a Gaussian mixture model (GMM) to the dominant allele frequencies to obtain the droplet clusters. The GMM may be a univariate GMM, which may be a skewed univariate GMM. A univariate GMM may be described using the probability density function in Equation 1:

$$p ( x ) = {\sum }_{k = 1}^{K} {π}_{k} 𝒩 ( x \vert {μ}_{k} {σ}_{k} ) , where K ∈ \{ 1 , 2 \} \qquad ( Equation 1 )$$

where K is the number of clusters in the data, π is the mixing proportion specifying the relative proportions of droplets in each cluster, μₖ is the mean, and σₖ is the standard deviation. Additionally, or alternatively, the clustering may be performed using K-means clustering, agglomerative clustering, density-based spatial clustering, or any other suitable clustering technique.

In embodiments where a GMM is fit to the dominant allele frequencies, an initial step may include determining the value of K in Equation 1. Any suitable techniques for determining the value of K may be used, as aspects of the technology described herein are not limited in this respect. For example, in some embodiments, Hartigan's dip test may be used to determine the value of K. Hartigan's dip test may be used to determine whether the distribution of dominant allele frequencies is unimodal or bimodal. The p-value for the test for unimodality may be used to determine the value of K. For example, if the p-value is greater than a threshold, the value of 1 may be selected for K. If the p-value is less than or equal to the threshold, the value of 2 may be selected for K. The threshold may include any suitable threshold such as, for example, 0.05, 0.15, 0.20, 0.25, 0.30, 0.35, any value between 0.05 and 0.4, or any other suitable threshold, as aspects of the technology described herein are not limited in this respect. Hartigan's dip test is described by Hartigan, J. & Hartigan, P (“The Dip Test of Unimodality.” Ann. Statist. 13 (1) 70-84, March 1985), which is incorporated by reference herein in its entirety.

In some embodiments, if the value of 1 is selected for K, this may indicate that all or almost all of the cells associated with the droplets are homozygous. In this scenario, the cells associated with the droplets may be genotyped as homozygous at the locus, and technique 100 may end. If the value of 2 is selected for K, then clustering of the dominant allele frequencies may proceed based on the number of clusters.

In some embodiments, after clustering the droplets into one or more clusters, one of the clusters is identified as the first set of droplets 110. In some embodiments, this includes identifying the cluster associated with the largest mean. In some embodiments, the first set of droplets 110 include droplets associated with cells that are homozygous at the locus. The other droplets 112 not included in the first set of droplets may include droplets associated with cells that are heterozygous at the locus and droplets encapsulating multiple cells.

At act 114, the scDNA-seq data 106 is used to identify a second set of droplets 116 from among the droplets 112 not included in the first set of droplets. In some embodiments, the second set of droplets 116 includes droplets associated with more than two alleles at the locus.

In some embodiments, identifying the second set of droplets 116 includes clustering the plurality of droplets into one or more droplet clusters and identifying one of the droplet clusters as the second set of droplets. In some embodiments, the clustering is performed based on ploidy scores for the droplets 112. In some embodiments, a ploidy score for a droplet is determined based on the allele counts of the second most common (e.g., minor allele) and third most common allele at the particular locus of a cell associated with the droplet. For example, the allele counts may be indicated by the scDNA-seq data 106. In some embodiments, the ploidy score is determined using Equation 2:

$$Ploidy score = {log}_{2} ( \frac{Allele 2}{Allele 3} ) \qquad ( Equation 2 )$$

In some embodiments, the clustering is performed using any suitable clustering technique, as aspects of the technology described herein are not limited in this respect. For example, in some embodiments, the clustering may be performed by fitting a Gaussian mixture model (GMM) to the dominant allele frequencies to obtain the droplet clusters. The GMM may be a univariate GMM. A univariate GMM may be described using the probability density function in Equation 1. Additionally, or alternatively, the clustering may be performed using K-means clustering, agglomerative clustering, density-based spatial clustering, or any other suitable clustering technique.

In embodiments where a GMM is fit to the ploidy scores, an initial step may include determining the value of K in Equation 1. Any suitable techniques for determining the value of K may be used, as aspects of the technology described herein are not limited in this respect. For example, in some embodiments, Hartigan's dip test may be used to determine the value of K. Hartigan's dip test may be used to determine whether the distribution of ploidy scores is unimodal or bimodal. The p-value for the test for unimodality may be used to determine the value of K. For example, if the p-value is greater than a threshold, the value of 1 may be selected for K. If the p-value is less than or equal to the threshold, the value of 2 may be selected for K. The threshold may include any suitable threshold such as, for example, 0.05, 0.15, 0.20, 0.25, 0.30, 0.35, any value between 0.05 and 0.4, or any other suitable threshold, as aspects of the technology described herein are not limited in this respect.

In some embodiments, if the value of 1 is selected for K, this may indicate that all or almost all of the droplets 112 are associated with droplets that either (a) are heterozygous or (b) encapsulate multiple cells, but only one or two alleles are detected for the locus. In this scenario, technique 100 may proceed to act 120. If the value of 2 is selected for K, then clustering of the ploidy scores may proceed based on the number of clusters.

In some embodiments, after clustering the droplets into one or more clusters, one of the clusters is identified as the second set of droplets 116. In some embodiments, this includes identifying the cluster associated with the smallest mean. In some embodiments, the second set of droplets 116 include droplets associated with more than two alleles at the locus. For example, these droplets may encapsulate more than one cell, causing more than two alleles to be detected for the locus. The other droplets 112 not included in the first set of droplets may include droplets associated with cells that are heterozygous at the locus and droplets encapsulating multiple cells but for which only one or two alleles are detected at the locus.

At act 120, the scDNA-seq data 106 is used to identify a third set of droplets 122 from among the droplets 118 not included in the first set of droplets 110 or the second set of droplets 116. In some embodiments, the third set of droplets 118 includes droplets associated with cells that are heterozygous at the locus.

In some embodiments, identifying the third set of droplets 122 includes clustering the plurality of droplets into one or more droplet clusters and identifying one of the droplet clusters as the third set of droplets.

In some embodiments, the clustering is performed based on allele frequencies of common alleles at the locus and a noise vector. For example, in some embodiments, the clustering may be performed on data that indicates, for each droplet, values indicative of allele counts of one or more common alleles at the locus and a value indicative the frequency of rare alleles at the locus (e.g., a component of the noise vector). In some embodiments, dimensionality reduction can be performed on the allele count data for the common alleles and the noise vector to obtain data with reduced dimensions (e.g., one-dimension, two-dimensions, three-dimensions, etc.), and the clustering can be performed in the lower dimensional space. Any suitable dimensionality reduction techniques can be used as aspects of the technology described herein are not limited in this respect. Nonlimiting examples of dimensionality reduction techniques include principal component analysis, singular value decomposition, and independent component analysis.

In some embodiments, the noise vector is determined based on rare alleles at the locus. This may include, for example, summing read counts for all alleles for the locus across droplets and identifying alleles that account for less than a threshold portion of all read counts. The threshold portion may include any suitable threshold as aspects of the technology described herein are not limited in this respect. For example, the threshold may be 0.5%, 0.75%, 1%, 1.25%, 1.5%, 1.75%, 2%, 2.5%, 3%, between 0.5% and 5%, or another suitable threshold. In some embodiments, the noise vector is generated by determining, for each droplet, the sum of rare allele counts in the droplet.

In some embodiments, the clustering is performed using any suitable clustering technique, as aspects of the technology described herein are not limited in this respect. For example, in some embodiments, the clustering may be performed by fitting a Gaussian mixture model (GMM) to the dominant allele frequencies to obtain the droplet clusters. When clustering one-dimensional allele count data, the GMM may be a univariate GMM. A univariate GMM may be described using the probability density function in Equation 1. When clustering multi-dimensional allele count data, the GMM may be a multivariate GMM. A multivariate GMM may be described using the probability density function in Equation 3:

$$p ( x ) = {\sum }_{k = 1}^{K} {π}_{k} 𝒩 ( x \vert {μ}_{k} {Σ}_{k} ) , where K ∈ \{ 1 , 2 \} \qquad ( Equation 2 )$$

where K is the number of clusters in the data, π is the mixing proportion specifying the relative proportions of droplets in each cluster, μₖ is the mean, and Σₖ is the covariance matrix (e.g., of the principal components). Additionally, or alternatively, the clustering may be performed using K-means clustering, agglomerative clustering, density-based spatial clustering, or any other suitable clustering technique.

In embodiments where a GMM is fit to the allele frequency data, an initial step may include determining the value of K in Equation 1 (if the data is one dimensional) or Equation 2 (if the data is multi-dimensional). Any suitable techniques for determining the value of K may be used, as aspects of the technology described herein are not limited in this respect. For example, in some embodiments, Hartigan's dip test may be used to determine the value of K. Hartigan's dip test may be used to determine whether the allele frequency distribution is unimodal or bimodal. For example, this may include determining whether the first principal component is unimodal or bimodal. The p-value for the test for unimodality may be used to determine the value of K. For example, if the p-value is greater than a threshold, the value of 1 may be selected for K. If the p-value is less than or equal to the threshold, the value of 2 may be selected for K. The threshold may include any suitable threshold such as, for example, 0.05, 0.15, 0.20, 0.25, 0.30, 0.35, any value between 0.05 and 0.4, or any other suitable threshold, as aspects of the technology described herein are not limited in this respect.

In some embodiments, if the value of 1 is selected for K, this may indicate that all or almost all of the cells associated with the droplets are heterozygous. In this scenario, the cells associated with droplets 118 may be genotyped as heterozygous at the locus, and technique 100 may end. If the value of 2 is selected for K, then clustering of the low dimensional data may proceed based on the number of clusters.

In some embodiments, after clustering the droplets into one or more clusters, one of the clusters is identified as the third set of droplets 122. In some embodiments, this includes (a) computing the determinant of E for each cluster to identify the cluster with the highest droplet density (e.g., smallest determinant). The cluster associated with the highest density in low dimensional space may be identified as the third set of droplets 122. The other droplets 124 not included in the third set of droplets 122 may include droplets encapsulating multiple cells.

In some embodiments, the first set of droplets 110 and the third set of droplets 122 may be used to genotype cells associated with the droplets in the first and third sets of droplets to obtain cell genotypes 126. For example, cells associated with droplets in the first set of droplets 110 may be genotyped as cells that are homozygous at the locus. Cells associated with droplets in the third set of droplets 122 may be genotyped as cells that are heterozygous at the locus.

In some embodiments, the cell genotypes 126 may be used to regulate treatment of a biological sample, such as biological sample 102 or a different biological sample. For example, in some embodiments, if the cell genotypes 126 may be used to inform whether to expand and use cells in the biological sample 102 to develop a treatment for one or more subjects or whether to discard the biological sample 102. Additionally, or alternatively, the cell genotypes 126 may inform modifications to the gene-editing and/or sequencing processes used for processing a subsequent biological sample.

### EXAMPLES

### Example 1: A Computational Workflow and Data Resource for Genotyping Single Cells from Gene Editing Experiments

In this example, a deep characterization of data derived from scDNAseq of CRISPR-Cas9 edited samples was performed and a computational workflow tailored to evaluate allelism in the context of gene editing was developed. Specifically, a ‘ground truth’ data atlas was created by running scDNAseq on artificial cocktails formed by mixing edited HL-60 clones with pre-defined edited allele variants of CLEC12A and/or CD33, two markers within the hematopoietic myeloid lineage. This data resource was used to delineate technical artifacts that could confound downstream interpretation of editing allelism.

This resource was also leveraged to develop a computational workflow called GUMM (Genotyping Using Mixture Models). In some embodiments, GUMM systematically genotypes single droplets from scDNAseq data by fitting a series of Gaussian mixture models (GMMs) to allele read counts generated by CRISPResso2. CRISPResso2 is described by Clement, K et al. (CRISPResso2 provides accurate and rapid genome editing sequence analysis. Nat Biotechnol 37, 224-226 (2019)), which is incorporated by reference herein in its entirety. When applied to the ground truth dataset, GUMM was shown to rapidly evaluate allelism in single cells and accurately estimate the original clonal ratios of the artificial cocktails. The example provides both a novel bioinformatic solution and rich data resource for researchers in the gene editing community looking to engineer cells with complex genotypes.

### Results

Generation of a Gene Editing scDNAseq Data Resource

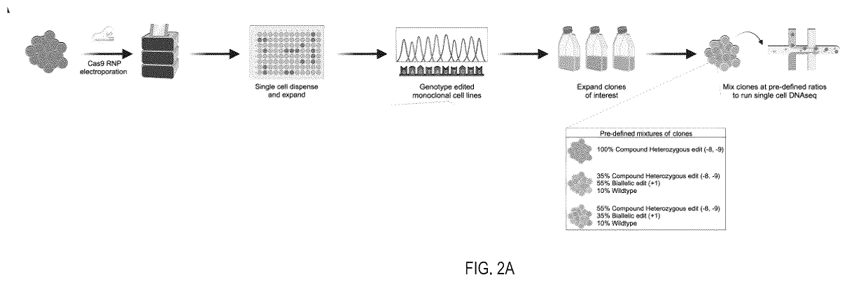

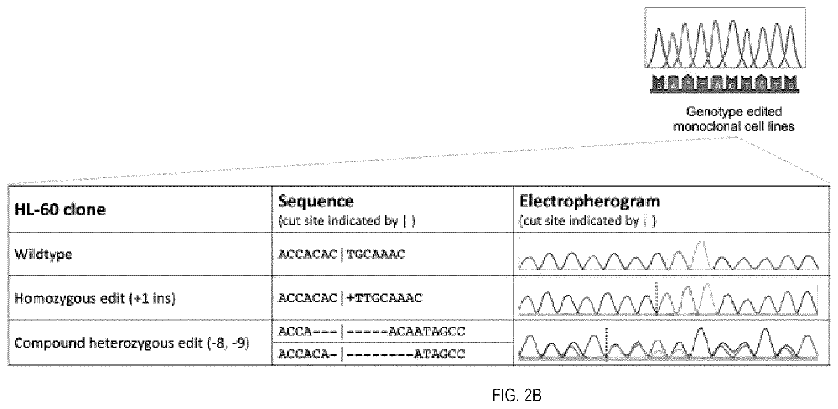

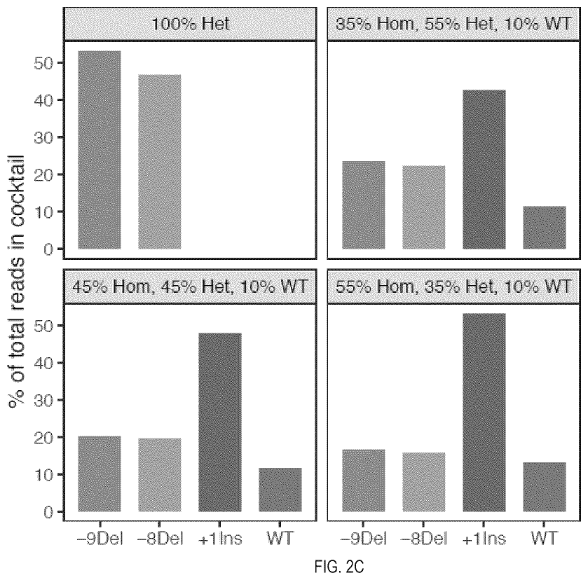

To create the data resource, CRISPR-Cas9 was employed to modify HL-60 cells at the CLEC12A gene, either individually or simultaneously with CD33. Singleplex and multiplex edited cells were isolated and expanded to create HL-60 monoclonal cell lines (FIG. 2A). Sanger sequencing and ICE analysis identified several singleplex edited clones edited at CLEC12A including one that was homozygous with a +1 insertion (+1Ins) and one that was heterozygous with a −8del and −9del compound deletion. ICE analysis is described by Conant et al. (Inference of CRISPR Edits from Sanger Trace Data. The CRISPR Journal 5, 123-130 (2022)), which is incorporated by reference herein in its entirety. One multiplexed clone homozygous for a +1 insertion at CLEC12A and homozygous for a −7Del at CD33 (FIG. 2B) was generated. Two artificial cocktails were generated by mixing singleplexed clones at varying ratios. The first comprised a mix of homozygous, heterozygous, and WT clones in proportions of 35%, 55%, and 10%, respectively. This sample was prepared with an additional filtering step that reduced the multiplet rate. Another two cocktails were created using the same clones but in proportions of 55%, 35%, and 10% and 45%, 45%, and 10%. These samples were not passed through the filtering step to produce data with a high multiplet rate. scDNAseq data with varying levels of noise was better modeled by altering the filtering protocol. Mission Bio's Tapestri platform was employed to generate barcoded single cell DNA libraries for these two cocktails, the singleplexed compound heterozygous clone, and the CLEC12A/CD33 multiplexed clone (FIG. 2A). After sequencing these libraries, the allele composition at the pseudobulk level was analyzed and the observed allele frequencies were consistent with the makeup of the cocktail (FIG. 2C).

FIGS. 2A-2D show the generation and validation of the artificial cocktails. FIG. 2A shows a graphical overview of an example procedure used to generate artificial mixtures of edited HL60 monoclones. FIG. 2B shows an electropherogram from Sanger sequencing of WT HL60 and two monoclones with homozygous and heterozygous editing of CLEC12A. Vertical line indicates cut site of CRISPR-Cas9. FIG. 2C shows bar plots indicating the total number of reads mapping to the four possible alleles across three cocktails. FIG. 2D shows example read alignment profiles of droplets from an artificial mix of cocktail.

Exploring Artifacts that Confound Allelism Interpretation

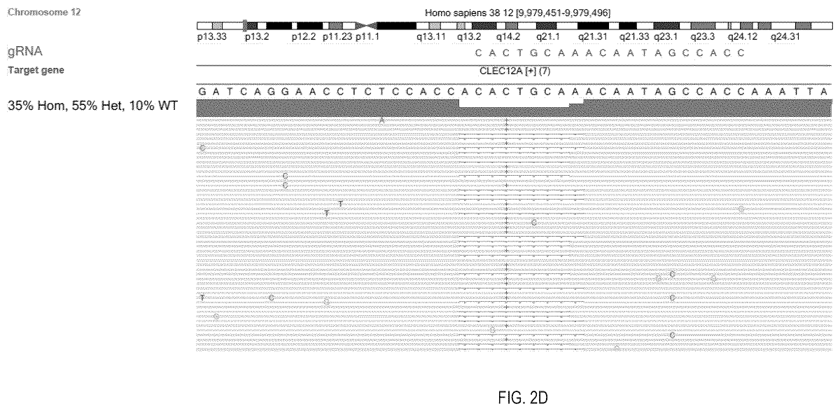

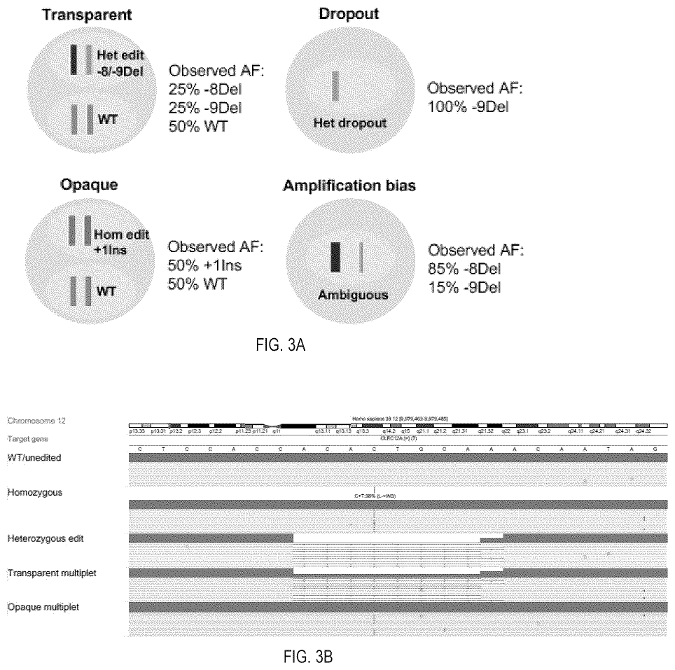

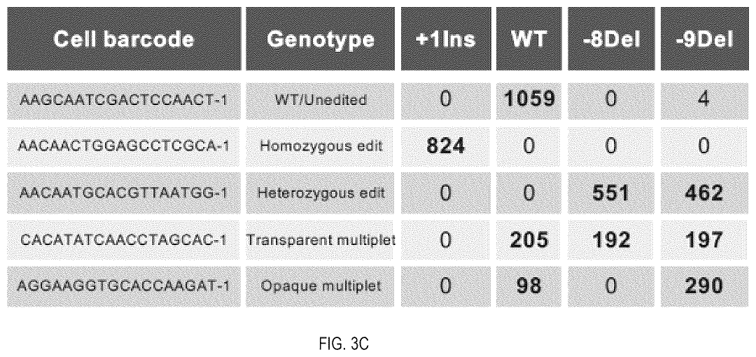

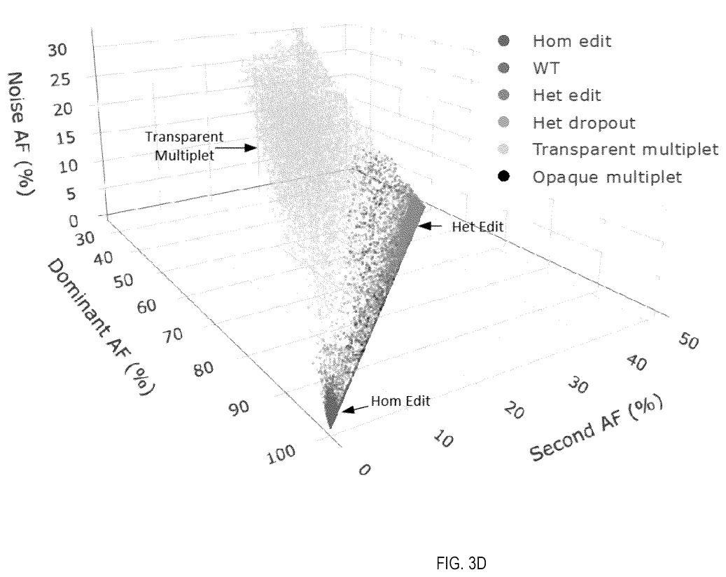

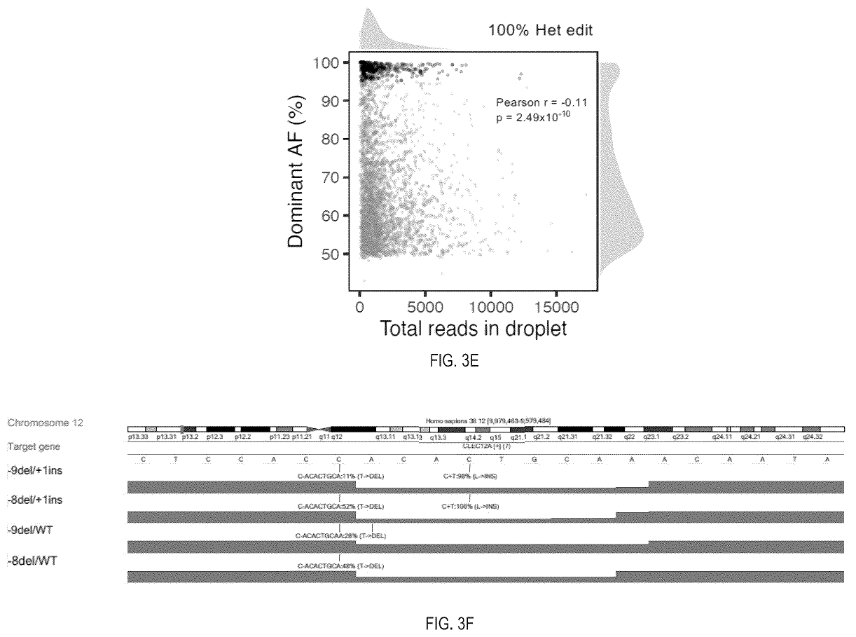

Because the singleplexed cocktails are comprised of pure clones with pre-defined editing patterns at the CLEC12A locus, the scDNAseq readout of allele frequencies was theoretically expected to reveal only droplets with 100% WT reads, 100% of reads containing the +1 Ins allele, or reads equally distributed between the −8Del and −9Del patterns. Indeed, these genotypes were observed in the data but several other interesting artifacts were noticed (FIG. 3A). First, droplets with more than two unique alleles indicating that two cells were encapsulated within the same droplet were observed. Droplets with ploidy greater than two were referred to as transparent multiplets (FIGS. 3B-3C). Droplets that displayed an allele frequency distribution consistent with that of a heterozygous cell but whose genotype cannot exist due to the clonal nature of the cocktails (e.g. WT/+1Ins) (FIGS. 3B-3C) were identified. These multiplets were referred to as opaque multiplets.

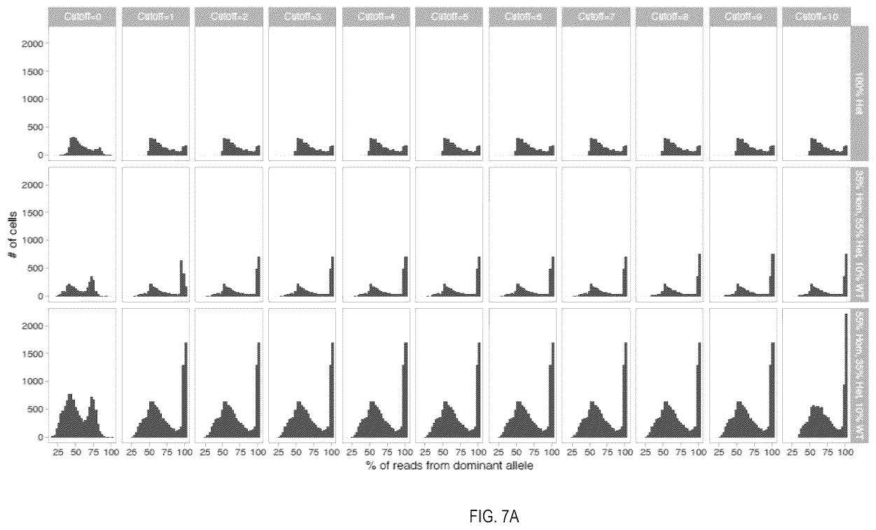

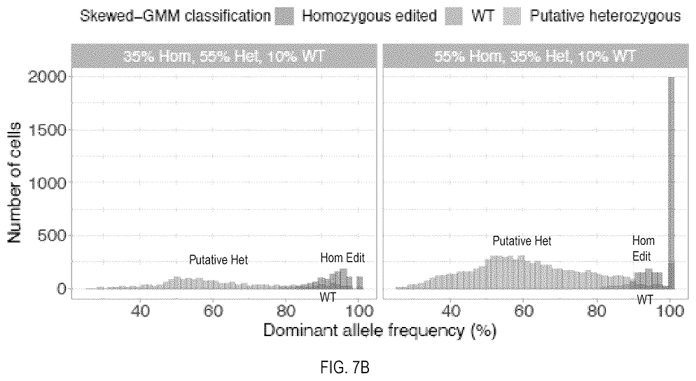

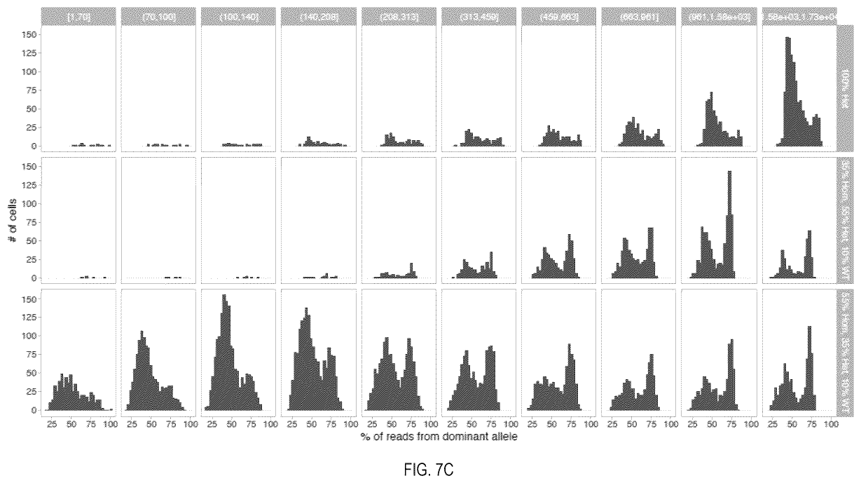

Second, the data show that heterozygous alleles deviated from a theoretical 50-50 allele frequency distribution, often displaying a bias towards one allele (FIG. 3D, FIG. 3E). This is most likely attributed to amplification bias, which may arise during PCR amplification of minimal DNA quantities, as is the case with droplet-based amplification. While this phenomenon may be anticipated in microfluidic technologies, it may introduce uncertainty when determining heterozygosity of a droplet. In addition, it was found that droplets with higher read depth at the CLEC12A editing site ameliorated some of this bias and improved the separability of homozygous and heterozygous droplets when analyzing their allele frequencies (FIG. 7C). Pre-processing of droplet allele frequencies may also improve the ability to resolve genotypes. For example, the dominant allele frequency of homozygous cells may approach 100% by removing alleles with less than 10 reads (FIGS. 7A-7C). However, this solution may not be viable for all scDNAseq experiments, as shown by prior studies that achieved an average sequencing depth of around 20 reads per droplet per amplicon. Thus, setting stringent filtering criteria may be counterproductive when analyzing less deeply sequenced libraries.

Moreover, dropouts in the heterozygous clone sample where the −8Del or −9Del allele was nearly undetectable from the readout leading to an erroneous homozygous droplet were observed. This occurred in 12.3% of the droplets and is within a range that may be expected for this platform. There was also a weak but significant association between dropout rate and droplet read depth (Pearson r=−0.11, p=2.49×10-10) (FIG. 3E, FIG. 7C). Multiplets and dropouts were not mutually exclusive as droplets with genotypes including +1Ins/−8Del, +1Ins/−9Del, WT/−8Del, and WT/−9Del were observed. These erroneous genotypes are consistent with a scenario where two cells are encapsulated in the same droplet with one cell experiencing allele dropout or amplification bias (FIG. 3F).

FIGS. 3A-3F show single cell characterization of artificial cocktails. FIG. 3A shows an illustration of possible artifacts in scDNAseq data and their resulting allele frequency readouts that confound interpretation of gene editing allelism. FIG. 3B shows read alignment profiles of six selected droplets from cocktails displaying different allele combinations observed at the CLEC12A edit site. FIG. 3C shows a table of allele read counts produced by CRISPResso2 for the six droplets. FIG. 3D shows a 3D scatterplot showing frequencies of each droplet's top three most frequent CLEC12A alleles across all cocktails. FIG. 3E shows a scatterplot showing relationship between read depth and dominant allele frequency of droplets from sample with 100% compound heterozygous edited cells. Read depth is the total number of reads in the droplet and dominant allele is the allele with the most reads mapped to it in the droplet. Black points indicate droplets with a dominant allele frequency of >95% which are considered dropouts. FIG. 3F shows read alignment profiles of four opaque multiplets with highly unlikely allele combinations.

FIG. 7A shows example histograms showing frequency of the dominant allele in cells in different artificial mixes of cells, according to some embodiments of the technology described herein. FIG. 7B shows example histograms showing frequency of the dominant allele in cells that were genotyped according to some embodiments of the technology described herein. FIG. 7C shows example histograms showing frequency of the dominant allele in cells in different artificial mixes of cells, according to some embodiments of the technology described herein.

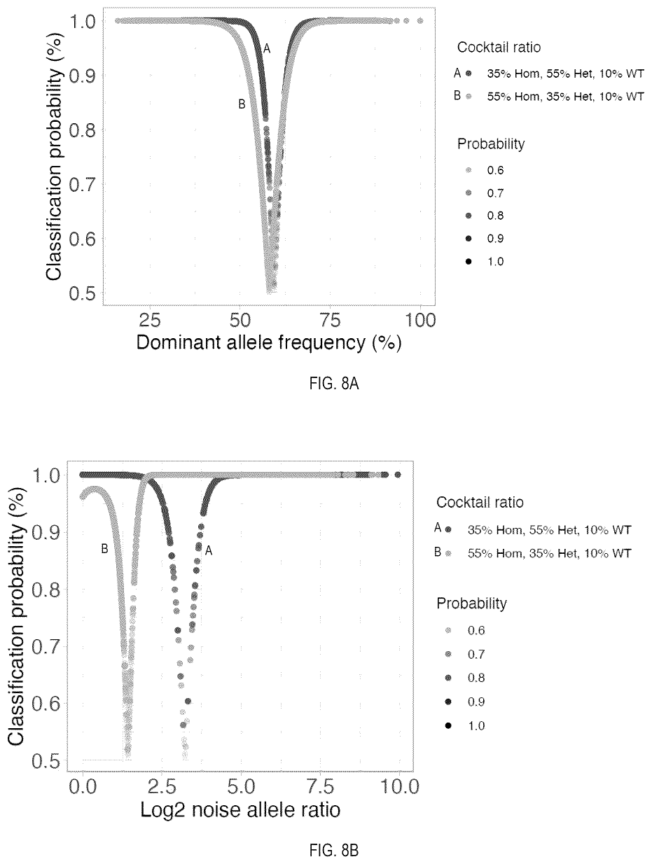

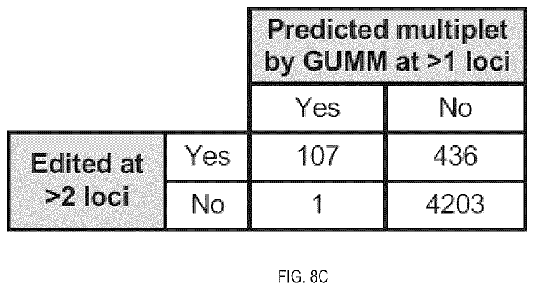

FIG. 8A shows an example of relationships between dominant allele frequency of a droplet and classification probability, according to some embodiments of the technology described herein. FIG. 8B shows an example of relationships between the log 2 noise allele ratio of a droplet and classification probability, according to some embodiments of the technology described herein. FIG. 8C shows an example table showing that genotyping cells in samples with a single gene-editing targets, according to embodiments of the technology described herein, can be used to identify droplets associated with multiple cells.

Automated Genotyping with Gaussian Mixture Models

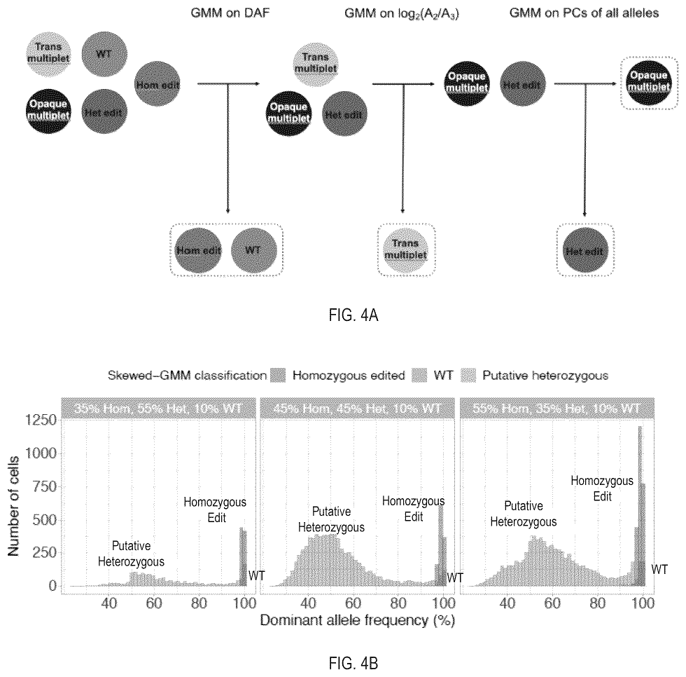

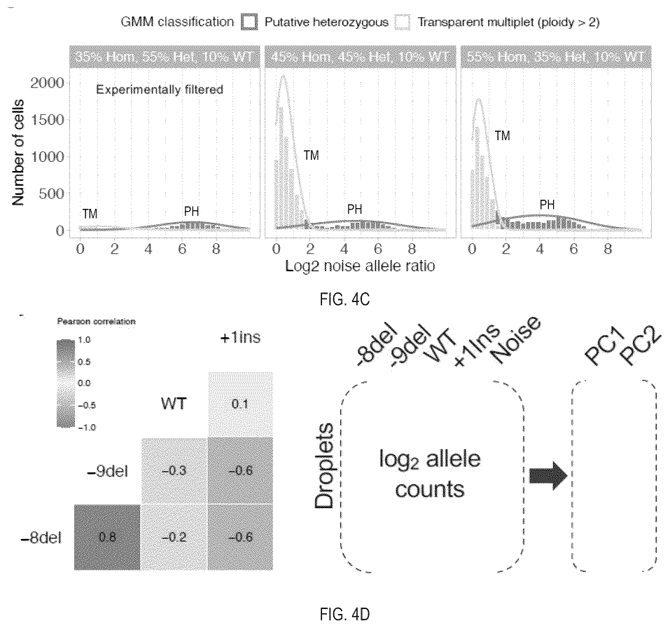

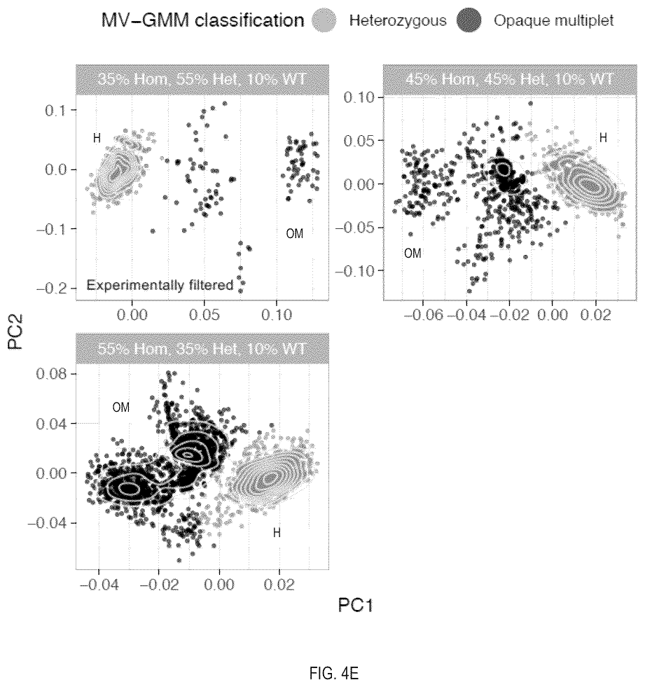

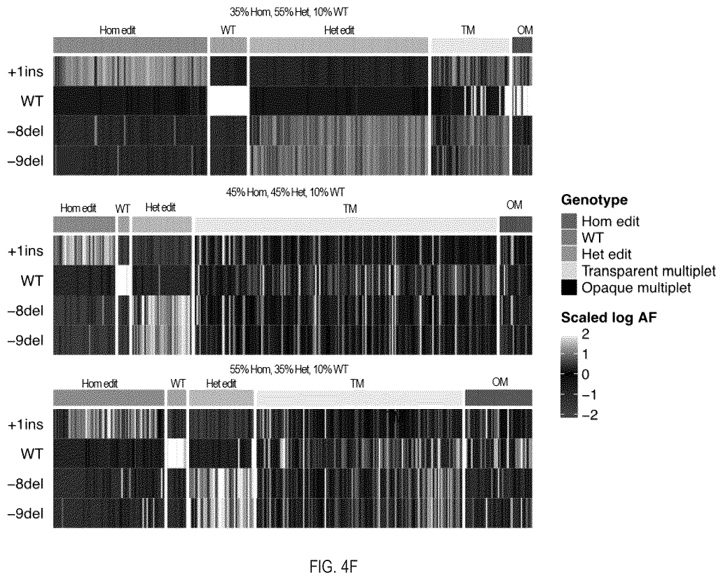

In this example, a systematic computational workflow named GUMM was developed that, in some embodiments, performs automated artifact-aware genotyping by fitting a series of Gaussian mixture models to the allele frequency readout of single droplets in a stepwise fashion. In some embodiments, GUMM involves identifying homozygous droplets, flagging transparent multiplets, and distinguishing heterozygous droplets from opaque multiplets. (FIG. 4A).

To evaluate the performance of GUMM, it was applied to the ground truth scDNAseq data generated from the artificial clonal cocktails starting with samples containing singleplexed edits. In the first step, GUMM successfully classified droplets into two distinct populations corresponding to homozygous and putative heterozygous droplets by fitting a skewed univariate GMM to read counts of the dominant CLEC12A allele in each droplet (FIG. 4B). No pre-filtering of low count alleles was performed on droplets to demonstrate that GUMM is robust to amplification bias and sequencing error. The same analysis was performed on pre-filtered data with similar results (FIG. 7B). Moreover, GUMM calculated the probability of each droplet's membership within the identified clusters, enabling the exclusion of droplets with genotype predictions of low confidence (FIGS. 8A-8B). In the second step, GUMM leveraged the ploidy information of each droplet to identify those that have more than two CLEC12A alleles. The ploidy of a droplet may be summarized by taking the log 2 ratio between the counts of the second most common CLEC12A allele and those of the third most frequently occurring CLEC12A allele, also referred to as the noise allele. The distribution of this noise allele ratio statistic unveiled two distinct populations both of which were effectively clustered by GUMM (FIG. 4C). The cluster with the lowest average log 2 noise allele ratio corresponded to transparent multiplets and displayed a triploid allele frequency distribution (FIG. 4C). The remaining pool of unclassified droplets were comprised of true heterozygous cells and opaque multiplets which occur when two homozygous cells (e.g. WT/+1ins) are enclosed in the same droplet yielding an allele frequency distribution consistent with that of a heterozygous cell. In order to distinguish these multiplets from true heterozygous cells, it was postulated that true heterozygous cells would include alleles that co-occur frequently across droplets. Indeed, this association was observed in the ground truth data where the −8Del allele is strongly correlated with the −9Del allele (Pearson r=0.81, p<<0.01) (FIG. 4D). Likewise, the heterozygous alleles were anti-correlated with the +1ins and WT alleles (FIG. 4D). GUMM captured this correlation structure in the remaining droplet pool by implementing principal component analysis (PCA) on the allele counts of the most frequently occurring alleles and the collapsed noise feature. On a dataset where homozygous and heterozygous alleles were not known a priori, the counts of all alleles were summed and a loose frequency cutoff (e.g 1-5%) was set to select common alleles for PCA. As the final step, GUMM fit a multivariate GMM to the first two principal components (PCs) to effectively cluster heterozygous cells separately from opaque multiplets (FIG. 4E). Inspection of each droplet's predicted genotype and their corresponding allele frequencies shows consistency with ground truth (FIG. 4F). In contrast, simple hierarchical clustering of the allele frequencies cannot achieve the same accuracy and resolution of GUMM (FIG. 9).

FIGS. 4A-4F shows automation of single cell genotyping using GUMM. FIG. 4A shows the example GUMM workflow which automates both genotyping and multiplet detection by fitting three separate Gaussian mixture models in series. FIG. 4B shows histograms showing frequency of the dominant CLEC12A allele in droplets for each genotype class predicted by fitting a skewed univariate GMM. FIG. 4C shows histograms of log 2 noise allele ratios for droplets with >2 detected CLEC12A alleles. Color indicates droplets predicted to be transparent multiplets or putative heterozygous edited droplets after fitting a univariate GMM. FIG. 4D shows a heatmap showing correlation of each of four possible CLEC12A alleles across putative heterozygous droplets. PCA was used to reduce log 2 read frequencies of all alleles into two dimensions. Read frequencies of rare alleles are summed into a single “noise” feature prior to dimensionality reduction. FIG. 4E shows scatterplots with density contours showing putative heterozygous cells in low dimensional space with distinct clusters corresponding to true heterozygous singlets and opaque multiplets. Color shows automated classification of clusters by fitting a multivariate GMM to PCs. FIG. 4F shows heatmaps showing scaled log frequencies of the four CLEC12A alleles across droplets. Top annotation bar indicates the CLEC12A genotype category each droplet was assigned to by GUMM.

FIG. 9 shows example heatmaps showing scaled log frequencies of four alleles at a locus across droplets associated with different artificial mixes of cells, according to some embodiments of the technology described herein.

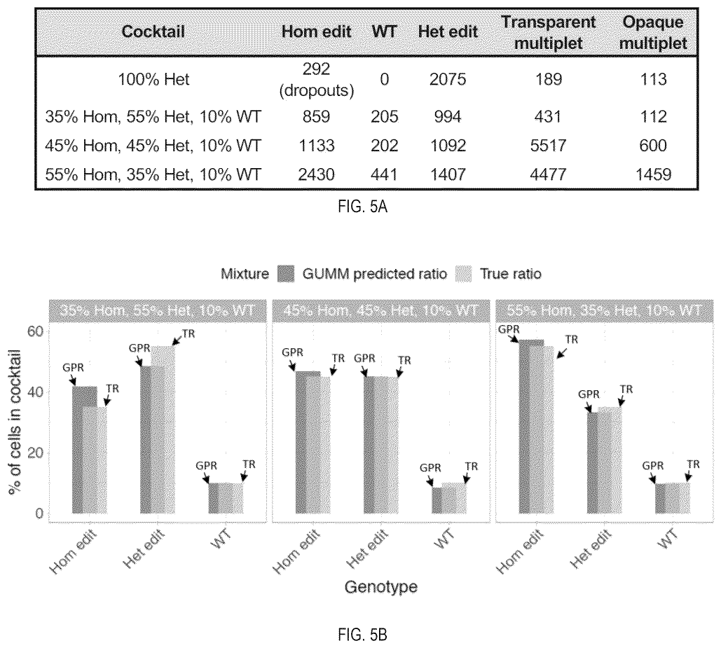

After determining allelism at the single cell level and identifying multiplets, GUMM estimated the sample composition by tallying the droplet genotypes after removing multiplets or splitting them into partial droplets. In the analysis, all multiplets were removed and it was found that GUMM was able to accurately estimate the original clonal fractions of the cocktails with less than 10% deviation in the 35% Hom, 55% Het, 10% WT cocktail and less than 5% deviation in the 55% Hom, 35% Het, 10% WT and 45% Hom, 45% Het, 10% WT cocktails (FIG. 5B). Although the 35% Hom, 55% Het, 10% WT cocktail contained less multiplets, a larger deviation from ground truth was observed.

FIGS. 5A-5B show estimating clonal composition of artificial cocktails. FIG. 5A shows a table of droplet counts in each genotype category across singleplex and multiplex cocktails. FIG. 5B shows bar plots comparing sample composition estimated by GUMM with ground truth for the 35% Hom, 55% Het, 10% WT, 45% Hom, 45% Het, 10% WT, and 55% Hom, 35% Het, 10% WT cocktails.

Analysis of Public scDNAseq Gene Editing Data

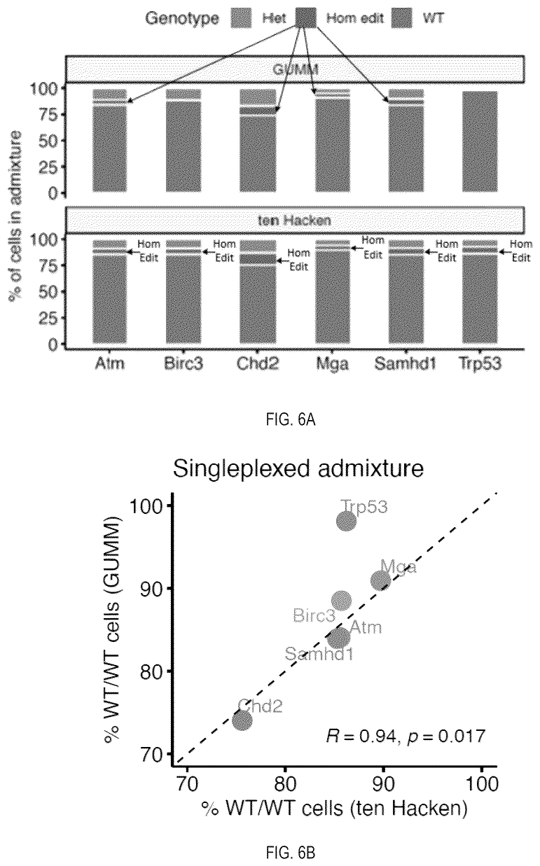

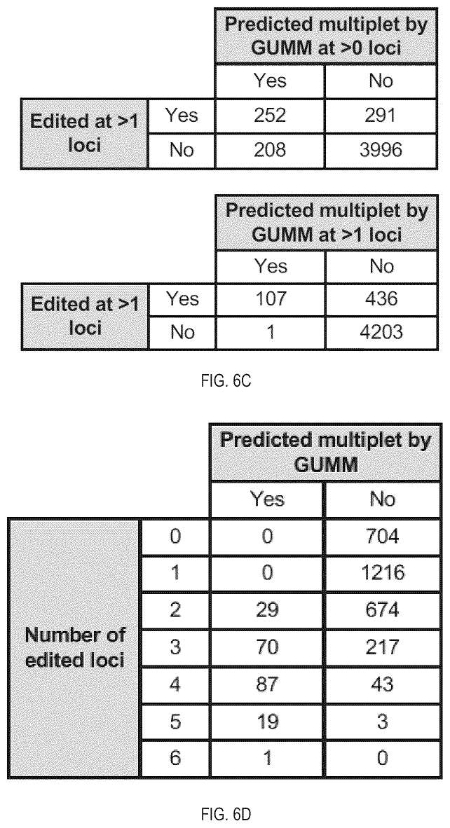

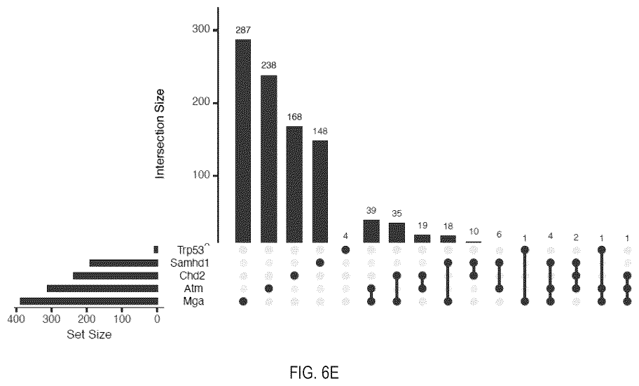

To demonstrate that GUMM could be applied across different datasets, it was applied to a published scDNAseq dataset generated from Ba/F3 mouse cells edited across six genes (Atm, Birc3, Chd2, Mga, Samhd1, Trp53) with CRISPR-Cas9. The first sample consisted of an admixture of singleplex edited Ba/F3 cells which enabled an orthogonal approach for identifying multiplets. Cells harboring multiple edited loci may be more likely to be multiplets. In this sample, GUMM was able to identify transparent multiplets but not opaque multiplets due to the allelic heterogeneity and the relatively low composition of diploid droplets, violating the assumption that most heterozygous cells comprise of two co-occurring alleles. Despite this limitation, it was still possible to predict sample genotype composition across the six genes and found that the estimates were concordant with the published results which adopted hard allele frequency threshold cutoffs (FIG. 6A). Since this sample consisted of primarily WT cells at any given gene, the WT cell composition estimates were compared with the published results and strong correlation across the six genes was observed (Spearman r=0.94, p=0.02) (FIG. 6B). The two orthogonal approaches for multiplet identification were compared and it was found that GUMM flagged 46.4% of cells with multigene edits as transparent multiplets and 95.1% of cells with a single gene edit as singleplets. Moreover, the stringency of a droplet being identified as transparent multiplet was increased by using evidence from at least two genes. At this stringency it was found that 19.7% of droplets with multigene edits were flagged as transparent multiplets and nearly 100% of droplets with editing at one gene were labeled as singleplets. These results suggest that in a true gene editing experiment where all cells are intended to harbor edits at the same genes, GUMM will still be able to identify a significant portion of multiplets in the data by just analyzing allele variants at each individual locus.

Next, GUMM was applied to the multiplex sample data where Ba/F3 cells were transduced with a pool of lentivirus expressing sgRNAs to simultaneously edit the six genes. It was found that droplets with no editing or editing at just one gene were unlikely to be flagged as a multiplet by GUMM (FIG. 6D). Droplets with an increasing number of edited genes were more likely to be flagged as multiplet which is consistent with the presumption that cells are less likely to receive multiple sgRNAs in a pooled transduction experiment (FIG. 6D). After removing multiplets, it was found that 136/3063 cells contained homozygous edits at more than one gene suggesting that a pooled lentivirus approach may not be useful for efficient multiplex editing (FIG. 6E).

FIGS. 6A-6E show an example application of GUMM to the public scDNAseq data. FIG. 6A shows the comparison of reported sample composition with GUMM's estimate from scDNAseq of singleplexed Ba/F3 sample. FIG. 6B shows a scatterplot showing correlation between the percent of WT/WT (unedited) cells estimated by GUMM and the percent reported in original study. Black diagonal dotted line indicates perfect concordance. FIG. 6C shows tables showing overlap between droplets being flagged as transparent multiplets by GUMM and those with editing at more than 1 gene in the singleplexed Ba/F3 sample. FIG. 6D shows a table showing the overlap between droplets identified as multiplets by GUMM and those with simultaneous edits across the six genes in the multiplexed Ba/F3 sample. FIG. 6E shows an upset plot showing number of cells with different combinations of homozygous gene edits.

### Methods

### CRISPR-Cas9 Editing and Expansion of HL60 Monoclones

HL-60 cells (CCL-240TM, ATCC) were cultured in 20% FBS in Iscove's Modified Dulbecco's Medium (IMDM, Cat. No. 12440053, ThermoFisher Scientific). Cas9-RNPs were delivered via electroporation using the Lonza Amaxa 4D-Nucleofector System (Cat No. AAF-1002, Lonza Bioscience) to 1e6 HL-60 cells in 100 uL 4D-Nucleofector Single Cuvettes (Cat. No. AXP-1003, Lonza Bioscience) with the SF Cell Line 4D-Nucleofector X Kit L (Cat. No. V4XC-2012, Lonza Bioscience). Post-electroporation, cells recovered in culture for 48 hours. Edited cells were single cell dispensed into one well of a flat bottom, tissue culture treated 96-well plate with 100 μL of 20% FBS in IMDM using the Namocell Hana Single Cell Dispenser (Cat. No. NI004, Namocell). Cells were expanded to confluency and genotyped using Sanger sequencing followed by ICE Analysis.

### Generation and Validation of Cocktails

Monoclonal singleplex or multiplex edited HL-60 cell lines were mixed at defined proportions with one another and unedited HL-60 cells that were cultured for the same amount of time as the monoclonal edited cells. To generate the cocktails, each cell line was counted in duplicate using the Nexcelom Cellometer (Auto 2000, Nexcelom) and the average total number of cells was used to calculate the number of cells to add to the mixture. Cocktails were generated immediately before running the MissionBio Tapestri protocol.

scDNAseq of Artificial Cocktails

Barcoded single cell libraries were produced for each cocktail using Mission Bio's Tapestri platform and a panel of 21 amplicons including two that covered CLEC12A and CD33 editing sites. Sample preparation was performed using Mission Bio's recommended protocol. Cells from the 35% Hom, 55% Het, 10% WT sample were filtered with a 40 uM Flomi before cell encapsulation to generate data with a low multiplet rate. Cells from the 55% Hom, 35% Het, 10% WT and 45% Hom, 45% Het, 10% WT samples were not filtered to generate data with a high multiplet rate. Cocktail libraries were sequenced on Illumina's NextSeq 2000 with a P2 600 cycle kit.

Analysis of scDNAseq Gene Editing Data Resource

Raw fastq files from each cocktail were processed using the command line implementation of the Tapestri Pipeline (v2.0.2) which performs QC, read trimming, alignment to the reference genome (GRCh38.p14), and barcode deconvolution. The summary report produced by the pipeline was used to assess amplicon uniformity, proper read alignment, and coverage. The pipeline outputs BAM files with each read assigned to a cell barcode under the read group (RG) tag. All BAM files were manually inspected on the genome browser (Qiagen OmicSoft Studio V11.2) to confirm the presence of expected editing patterns at the pseudobulk and single cell level. To quantify editing, each BAM file was split into individual cell-level BAM files with bamtools and an inhouse script was used to run CRISPResso2 in parallel on individual cell in “WGS” mode with the following parameters: —quantification_window_center-3—quantification_window_size 10—min_reads_to_use_region 5—demultiplex_only_at_amplicons—ignore_substitutions—exclude_bp_from_left 1—exclude_bp_from_right 1. The target amplicon regions are slim to the regions of spacer guide RNA with either ±30 bp flanking regions for the internal and public datasets to reduce the effect of variant read length and increase computational efficiency. The output for each cocktail was concatenated into a single table summarizing allele read counts for each barcode. Detailed information on each allele was provided including editing status, DNA sequence, and indel profile. Barcodes with <10 total counts were removed from the data prior to genotyping and sample composition estimation. Prior to GUMM analysis, allele data were collapsed by ignoring substitutions and summing up their counts. Allele frequencies for each cell were calculated by dividing the number of counts from an allele by the total number of counts across all alleles per locus per cell.

### Gaussian Mixture Modeling of Droplet Allele Frequencies and Sample Composition Estimation

In this example, GUMM workflow involved fitting a series of three GMMs to allele frequency distributions in a stepwise manner to genotype individual cells and flag multiplets. The GMMs were applied to classify droplets based on transformations of their allele counts. The GMMs may be described by the following probability density functions:

$$p ( x ) = {\sum }_{k = 1}^{K} {π}_{k} 𝒩 ( x \vert {μ}_{k} {σ}_{k} ) , where K ∈ \{ 1 , 2 \}$$

for the univariate case and

$$p ( x ) = {\sum }_{k = 1}^{K} {π}_{k} 𝒩 ( x \vert {μ}_{k} {Σ}_{k} ) , where K ∈ \{ 1 , 2 \}$$

for the multivariate case. K is the number of components or clusters in the data. K was restricted to 1 or 2. π is the mixing proportion specifying the relative proportions of droplets in each cluster.

In this example, GUMM began by fitting a skewed univariate GMM to the dominant allele frequencies calculated for all droplets using one or two mixing components (i.e. k=1, 2). In this example, the skewed models were found to be more accurate when identifying homozygous droplets because dominant allele frequencies of droplets are biased towards 100%. Hartigan's dip test was used on the ploidy scores to determine K where:

$$K = \{ \begin{matrix}1 , & p > 0.2 \\ 2 , & p \leq 0.2\end{matrix}$$

A K=1 model indicates the sample is homogenous (e.g. 100% edited or WT) and a K=2 model indicates a sample is heterogeneous. If K=2, the cluster corresponding to homozygous cells was identified by:

k_{Homozygous}=argmax[μ₁,μ₂]

The remaining cells were then used as input for the second step where GUMM flagged transparent multiplets by analyzing the ploidy of these non-homozygous droplets at the target locus. Ploidy was assessed by taking the log 2 allele count ratio of the 2nd and 3rd most common alleles in each droplet. This ratio was referred to as the ploidy score:

$$Ploidy score = {log}_{2} ( \frac{Allele 2}{Allele 3} )$$

Droplets with only two detectable alleles were whitelisted as true heterozygous cells and were not modeled. A univariate GMM was then fit to the ploidy scores to classify the remaining droplets where x is the ploidy score of the ith droplet and μₖ and σₖ are the mean and standard deviation of the ploidy score, respectively, of the kth component. Again, Hartigan's dip test was used to determine K. If K=2, the cluster with the smallest mean ploidy score was labeled as transparent multiplets:

7k_{Transparent multiplet}=argmin[μ₁,μ₂]

In the third and final step of this example, GUMM classified the final pool of droplets as either true heterozygous cells or opaque multiplets. It was assumed that two alleles that strongly co-occur comprise the genotype of true heterozygous cells. Likewise, droplets with rare allele combinations were assumed to most likely be erroneous diploid multiplets consisting of two homozygous cells encapsulated in the same droplet (i.e. WT and +1Ins). To leverage this information, GUMM summed up read counts for all alleles across droplets and identified rare alleles that account for <1% of all read counts. Rare alleles were collapsed into a single noise vector by summing up their counts in each droplet (FIG. 4D). GUMM then performed principal component analysis (PCA) on the allele frequencies of the common alleles and the noise vector. A multivariate GMM was then fit to the first two principal components (PCs) of the data to identify droplet clusters. Hartigan's dip test was performed on the first PC to determine K. If K=2, μₖ was a vector containing the PC means and Σₖ was the covariance matrix of the PCs. The determinant of Σ was computed for each cluster to identify the one with highest droplet density (smallest determinant). The cluster with higher density in low dimensional space corresponded to true heterozygous droplets:

k_{Heterozygous}=argmin[Det(Σ₁),Det(Σ₂)]

The skewed univariate, univariate, and multivariate GMMs were implemented using the mixsmsn and mclust R packages, respectively. Mixmsn is described by Prates et al. (mixsmsn: Fitting Finite Mixture of Scale Mixture of Skew-Normal Distributions. J. Stat. Soft. 54, (2013)) and mclust is described by Scrucca et al. (mclust 5: Clustering, Classification and Density Estimation Using Gaussian Finite Mixture Models. The R Journal 8, 289 (2016)), each of which is incorporated by reference herein in its entirety.

Analysis of Public scDNAseq Data

scDNAseq data was downloaded from Sequence Read Archive (SRA) under Bioproject accession number PRJNA665752. The data included a Ba/F3 sample consisting of an admixture of cells edited at only one of the following loci: Trp3, Birc3, Atm, Chd2, Mga, or Samhd1. It also included a multiplexed Ba/F3 sample where multiple edits were present in the same cell. Both samples were processed using the same procedure and parameters as with the artificial cocktails except reads were instead aligned to Gencode's mm10 (GRCm38.p4) mouse reference genome and CRISPResso2 was run using WGS mode with a flank parameter of 15 bp. In the singleplexed sample, droplets with less than 120 total reads, corresponding to approximately 20 reads per gene, were removed from the CRISPResso2 output.

### Example 2: GUMM: A Purpose-Built Computational Workflow for Single Cell Genotyping of Gene Editing Experiments

In this example, a novel computational workflow called GUMM (Genotyping Using Mixture Models) was developed that systematically infers single cell allelism at select loci from scDNAseq data by fitting a series of Gaussian mixture models (GMMs) to allele read counts generated by CRISPResso2. Among other applications, GUMM was shown to be well-suited for analyzing CRISPR-Cas9 gene editing experiments where cells in the sample are genetically homogenous and differ only at the intended editing site(s). GUMM output a probabilistic prediction of cell genotype and addressed technical artifacts including low coverage at the editing site, PCR amplification imbalance, multiplets, and sequencing error. Moreover, a gene editing “ground truth” scDNAseq atlas was developed to deeply characterize these technical artifacts and was leveraged in developing GUMM.

In this example, a computational workflow called GUMM was developed to rapidly genotype scDNAseq data from gene editing experiments. The method was applied to artificial mixtures of CRISPR-Cas9 edited HL-60 clones with distinct allele combinations at a single target gene. The workflow accurately genotyped individual cells based purely on various transformations of the allele frequency readout produced by CRISPResso2. It remained robust to data containing technical artifacts including amplification bias and multiplet contamination. The study provided both a rich data resource and novel bioinformatic solution for researchers in the gene editing community looking to characterize complex genotypes in engineered cell populations.

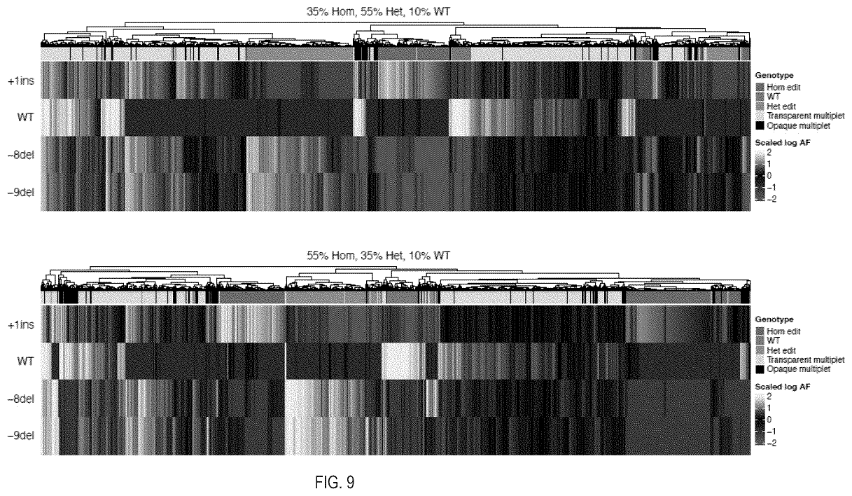

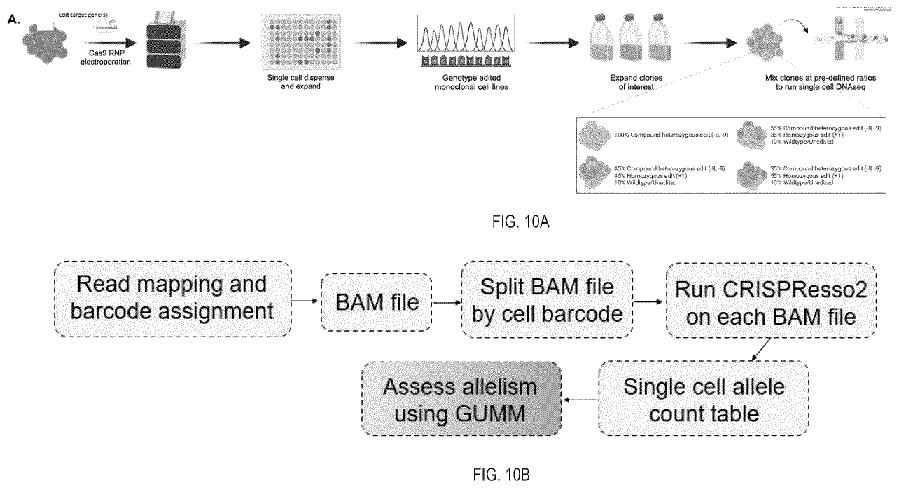

FIGS. 10A-10B show the construction of a “ground truth” gene editing scDNAseq resource and analysis workflow. FIG. 10A shows the CRISPR-Cas9 used to create edited HL-60 cells and expanded clones with distinct homozygous and compound heterozygous indel profiles. Clones were mixed at pre-defined ratios to create artificial cocktails that mimic the potential editing diversity of a CRISPR-Cas9 experiment. Three unique cocktails were sequenced to generate an atlas containing single cell readout for more than 20,000 cells. FIG. 10B shows an overview of an example computational pipeline used to analyze single cell DNA sequencing (scDNAseq) data from artificial cocktails.

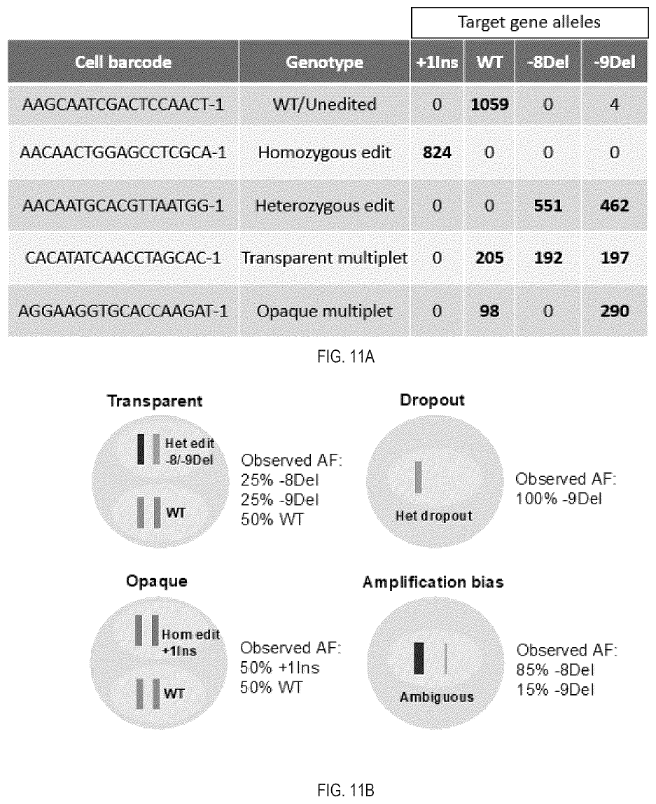

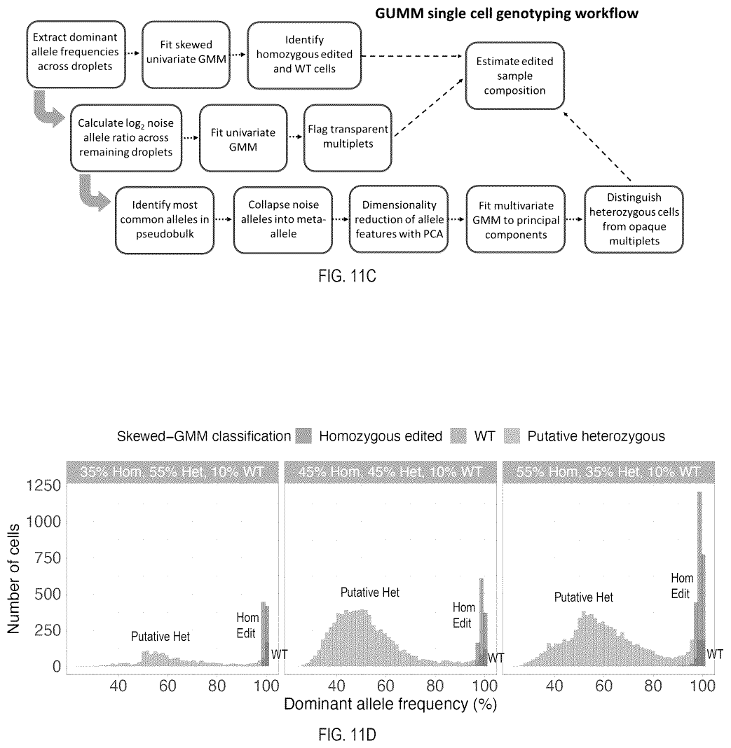

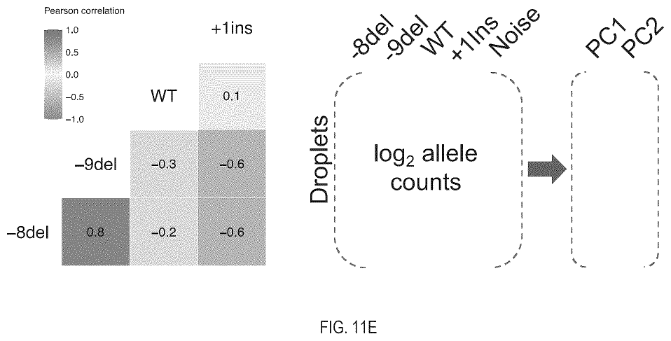

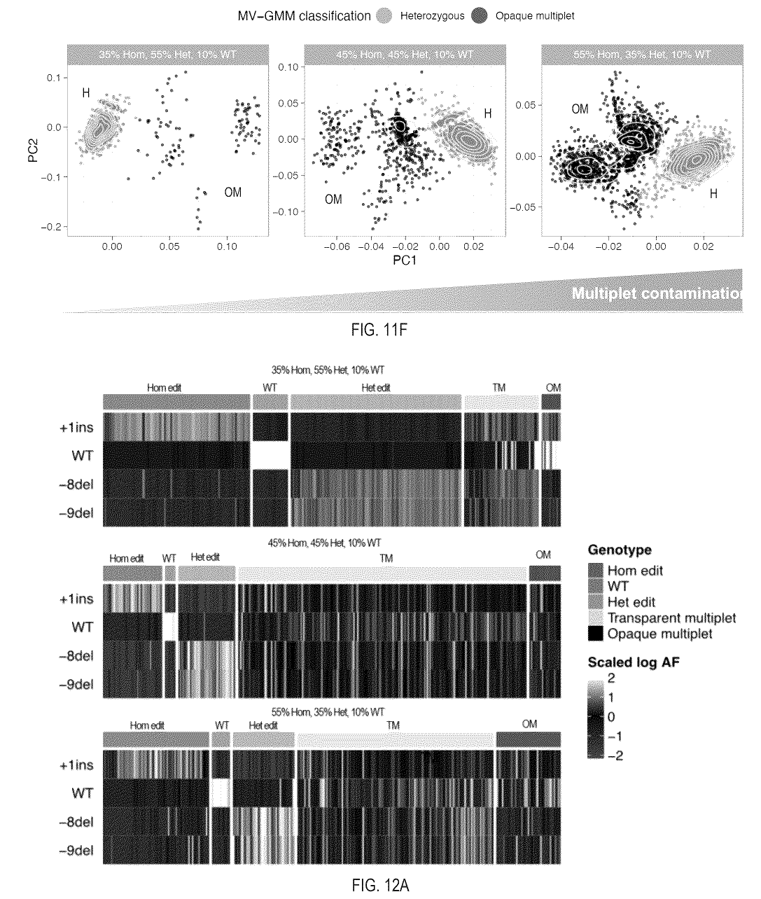

FIGS. 11A-11F show an example of artifact-aware single cell genotyping of artificial cocktails using GUMM. FIG. 11A shows CRISPResso2 output of allele counts for six distinct droplet types observed in “ground truth” data resource. Homozygous droplets were identified by a +1 insertion and heterozygous cells are characterized by a −8/−9 compound deletion. FIG. 11B shows an illustration of four potential artifacts observed in the scDNAseq data that may confound allelism assessment in gene editing experiments. Examples of allele frequencies (AF) corresponding to these scenarios are shown in FIG. 11B. FIG. 11C shows a diagram of an example GUMM workflow. In this example, the GUMM workflow included a series of Gaussian mixture models (GMM) used to identify homozygous cells, true compound heterozygous cells (Het edit), transparent multiplets (triploid), and opaque multiplets (diploid). The workflow started with allele read counts quantified by CRISPResso2 and automated the cell genotyping process to output cell-level genotype predictions and sample-level composition estimates. Multiplets were simultaneously flagged using ploidy information and principal component analysis (PCA) of allele counts. FIG. 11D shows the distribution of dominant allele frequency across all droplets and identification of homozygous cells by GUMM. scDNAseq protocol for 35% Hom, 55% Het, 10% WT cocktail included an additional cell filtering step prior to droplet barcoding to decrease multiplet rate. FIG. 11E shows a correlogram showing allele correlation structure revealing heterozygous alleles with high co-occurrence. PCA was performed on allele counts to generate low dimensional representation of data. FIG. 11F shows scatterplots showing opaque multiplet identification by fitting GUMM to first two principal components. Dense cluster of droplets correspond to true heterozygous cells with −8/−9 compound deletion.

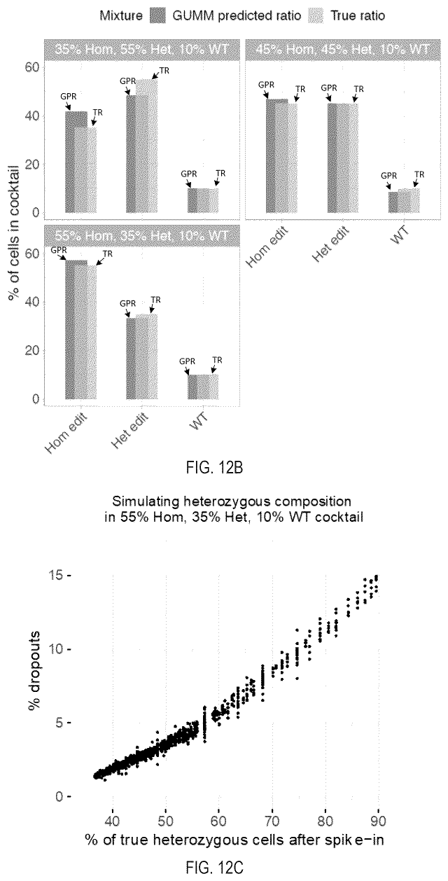

FIGS. 12A-12C show GUMM was used to accurately predict cell genotype and estimate original cocktail ratios. FIG. 12A shows heatmaps showing log-transformed frequencies of four possible alleles at edit site across droplets for all three artificial cocktails. Annotation bar indicates genotype or multiplet category predicted by GUMM. Allele frequency patterns of predicted genotypes are consistent with “ground truth” FIG. 12B shows bar plots comparing estimated and true cocktail genotype compositions after removing multiplets. The 35% Hom, 55% Het, 10% WT cocktail showed greater deviation from ground truth compared to other cocktails due to higher dropout rate which correlates with greater Het composition. FIG. 12C shows simulating heterozygous composition of 55% Hom, 35% Het, 10% WT cocktail by in silico spike-in. Cells were randomly selected from the pure 100% heterozygous sample data and computationally added to artificial cocktail data at increasing frequency. This was performed concurrently with cocktail down-sampling to increase the maximum heterozygous rate in the cocktail. Each random sampling event was repeated 5 times to ensure robustness. An association between cocktail heterozygous composition and dropout rate was observed in the simulation.

### Computer Implementation

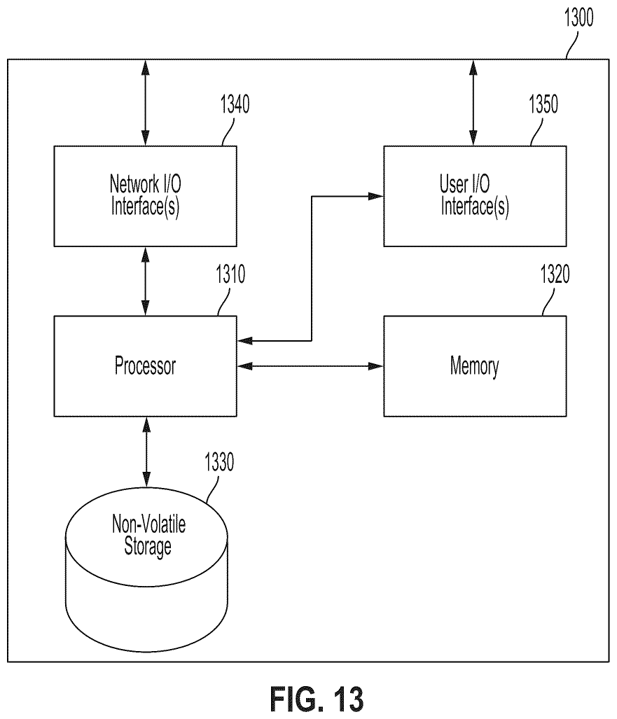

An illustrative implementation of a computer system 1300 that may be used in connection with any of the embodiments of the technology described herein is shown in FIG. 13. The computer system 1300 includes one or more processors 1310 and one or more articles of manufacture that comprise non-transitory computer-readable storage media (e.g., memory 1320 and one or more non-volatile storage media 1330). The processor 1310 may control writing data to and reading data from the memory 1320 and the non-volatile storage device 1330 in any suitable manner, as the aspects of the technology described herein are not limited to any particular techniques for writing or reading data. To perform any of the functionality described herein, the processor 1310 may execute one or more processor-executable instructions stored in one or more non-transitory computer-readable storage media (e.g., the memory 1320), which may serve as non-transitory computer-readable storage media storing processor-executable instructions for execution by the processor 1310.

Computing device 1300 may include a network input/output (I/O) interface 1340 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing device 1300 may also include one or more user I/O interfaces 1350, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### EQUIVALENTS AND SCOPE

Those skilled in the art will recognize, or be able to ascertain using no more than routine experimentation, many equivalents of the exemplary embodiments described herein. The scope of the present disclosure is not intended to be limited to the above description.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, it is to be understood that every possible individual element or subgroup of the elements is also disclosed, and that any element or subgroup of elements can be removed from the group. It is also noted that the term “comprising” is intended to be open and permits the inclusion of additional elements, features, or steps. It should be understood that, in general, where an embodiment, is referred to as comprising particular elements, features, or steps, embodiments, that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

All publications, patent applications, patents, and other references (e.g., sequence database reference numbers) mentioned herein are incorporated by reference in their entirety.

In addition, it is to be understood that any particular embodiment of the present disclosure may be explicitly excluded from any one or more of the claims. Where ranges are given, any value within the range may explicitly be excluded from any one or more of the claims. For purposes of brevity, all of the embodiments in which one or more elements, features, purposes, or aspects is excluded are not set forth explicitly herein.

1. A method for genotyping a plurality of cells in a biological sample using single-cell DNA sequencing (scDNA-seq) data obtained for a plurality of droplets, each of the plurality of droplets being associated with at least one cell of the plurality of cells, the method comprising:
using at least one computer hardware processor to perform:
obtaining the scDNA-seq data for the plurality of droplets, the scDNA-seq data having been previously obtained by sequencing the plurality of cells using scDNA-seq, wherein the scDNA-seq data comprises values indicative of frequencies of one or more alleles at a locus, the values including, for each particular droplet of the plurality of droplets, one or more values indicative of respective frequencies of the one or more alleles at the locus of a genome of at least one cell associated with the particular droplet; and
genotyping the plurality of cells using the scDNA-seq data to obtain a respective plurality of cell genotypes, the genotyping comprising determining, using the scDNA-seq data, for each particular droplet of the plurality of droplets, a genotype for the locus of the respective genome of the at least one cell associated with the particular droplet, the determining comprising:
identifying, using the scDNA-seq data and from among the plurality of droplets, a first set of droplets associated with cells that are homozygous at the locus;
identifying, using the scDNA-seq data and from among the plurality of droplets not in the first set of droplets, a second set of droplets associated with more than two alleles at the locus; and
identifying, using the scDNA-seq data and from among the plurality of droplets not in the first or second sets of droplets, a third set of droplets associated with cells that are heterozygous at the locus.

2. The method of claim 1, wherein the biological sample was previously processed using CRISPR-Cas9 gene editing.

3. The method of claim 1, further comprising:
processing the biological sample using CRISPR-Cas9 gene editing.

4. The method of claim 1, further comprising:
regulating treatment of a second biological sample based on the plurality of cell genotypes.

5. The method of claim 4, wherein regulating the treatment of the second biological sample comprises outputting, based on the plurality of cell genotypes, a recommendation for modifying a manner in which one or more materials are added to the second biological sample.

6. The method of claim 4, wherein regulating the treatment of the second biological sample comprises modifying a manner in which one or more materials are added to second biological sample.

7. The method of claim 1, further comprising:
regulating treatment of the biological sample based on the plurality of cell genotypes.

8. The method of claim 7, wherein regulating the treatment of the biological sample comprises outputting, based on the plurality of cell genotypes, a recommendation for expanding cells in the biological sample.

9. The method of claim 7, wherein regulating the treatment of the biological sample comprises expanding cells in the biological sample.

10. The method of claim 1, wherein identifying the first set of droplets comprises:
clustering the plurality of droplets into a first set of one or more droplet clusters; and
identifying a particular droplet cluster of the first set of one or more droplet clusters as the first set of droplets,
wherein clustering the plurality of droplets into the first set of one or more droplet clusters comprises clustering the plurality of droplets based on dominant allele frequencies for the plurality of droplets, wherein the dominant allele frequencies are specified by the scDNA-seq data.

11. The method of claim 10, wherein clustering the plurality of droplets comprises:
fitting a first Gaussian mixture model (GMM) to the dominant allele frequencies; and
using the fitted first GMM to obtain the first set of one or more droplet clusters.

12. The method of claim 1, wherein identifying the second set of droplets comprises:
clustering the plurality of droplets not in the first set of droplets into a second set of one or more droplet clusters; and
identifying a particular droplet cluster of the second set of one or more droplet clusters as the second set of droplets,
wherein clustering the plurality of droplets not in the first set of droplets comprises clustering the plurality of droplets not in the first set of droplets based on a respective plurality of ploidy scores for the plurality of droplets not in the first set of droplets.

13. The method of claim 12, further comprising:
determining the respective plurality of ploidy scores for the plurality of droplets not in the first set of droplets, the determining comprising determining the respective plurality of ploidy scores based on (a) minor allele counts for the plurality of droplets and (b) allele counts for a third most common allele for the plurality of droplets, wherein the minor allele counts and the allele counts for the third most common allele are specified by the scDNA-seq data.

14. The method of claim 12, wherein clustering the plurality of droplets not in the first set of droplets comprises:
fitting a second GMM to the ploidy scores; and
using the fitted second GMM to obtain the second set of one or more droplet clusters.

15. The method of claim 1, wherein identifying the third set of droplets comprises:
clustering the plurality of droplets not in the first or second sets of droplets into a third set of one or more droplet clusters; and
identifying a particular droplet cluster of the third set of one or more droplets clusters as the third set of droplets,
wherein clustering the plurality of droplets not in the first or second sets of droplets comprises clustering the plurality of droplets not in the first or second sets of droplets based on principal components of allele frequencies for the plurality of droplets not in the first or second sets of droplets, wherein the allele frequencies are specified by the scDNA-seq data.

16. The method of claim 15, further comprising:
performing dimensionality reduction on the allele frequencies to obtain the principal components of the allele frequencies.

17. The method of claim 15, wherein clustering the plurality of droplets not in the first or second sets of droplets based on the principal components of the allele frequencies comprises:
fitting a third GMM to the principal components of the allele frequencies; and
using the fitted third GMM to obtain the third set of one or more droplet clusters.

18. The method of claim 1, wherein droplets not in the first, second, or third sets of droplets are each associated with multiple cells of the plurality of cells.

19. The method of claim 1, further comprising sequencing the biological sample using scDNA-seq to obtain the scDNA-seq data.

20. A system, comprising:
at least one computer hardware processor; and
at least one non-transitory computer-readable storage medium storing processor-executable instructions that, when executed by the at least one computer hardware processor, cause the at least one computer hardware processor to perform a method for genotyping a plurality of cells in a biological sample using single-cell DNA sequencing (scDNA-seq) data obtained for a plurality of droplets, each of the plurality of droplets being associated with at least one cell of the plurality of cells, the method comprising:
obtaining the scDNA-seq data for the plurality of droplets, the scDNA-seq data having been previously obtained by sequencing the plurality of cells using scDNA-seq, wherein the scDNA-seq data comprises values indicative of frequencies of one or more alleles at a locus, the values including, for each particular droplet of the plurality of droplets, one or more values indicative of respective frequencies of the one or more alleles at the locus of a genome of at least one cell associated with the particular droplet; and
genotyping the plurality of cells using the scDNA-seq data to obtain a respective plurality of cell genotypes, the genotyping comprising determining, using the scDNA-seq data, for each particular droplet of the plurality of droplets, a genotype for the locus of the respective genome of the at least one cell associated with the particular droplet, the determining comprising:
identifying, using the scDNA-seq data and from among the plurality of droplets, a first set of droplets associated with cells that are homozygous at the locus;
identifying, using the scDNA-seq data and from among the plurality of droplets not in the first set of droplets, a second set of droplets associated with more than two alleles at the locus; and
identifying, using the scDNA-seq data and from among the plurality of droplets not in the first or second sets of droplets, a third set of droplets associated with cells that are heterozygous at the locus.

21. At least one non-transitory computer-readable storage medium storing processor-executable instructions that, when executed by at least one computer hardware processor, cause the at least one computer hardware processor to perform a method for genotyping a plurality of cells in a biological sample using single-cell DNA sequencing (scDNA-seq) data obtained for a plurality of droplets, each of the plurality of droplets being associated with at least one cell of the plurality of cells, the method comprising:
obtaining the scDNA-seq data for the plurality of droplets, the scDNA-seq data having been previously obtained by sequencing the plurality of cells using scDNA-seq, wherein the scDNA-seq data comprises values indicative of frequencies of one or more alleles at a locus, the values including, for each particular droplet of the plurality of droplets, one or more values indicative of respective frequencies of the one or more alleles at the locus of a genome of at least one cell associated with the particular droplet; and
genotyping the plurality of cells using the scDNA-seq data to obtain a respective plurality of cell genotypes, the genotyping comprising determining, using the scDNA-seq data, for each particular droplet of the plurality of droplets, a genotype for the locus of the respective genome of the at least one cell associated with the particular droplet, the determining comprising:
identifying, using the scDNA-seq data and from among the plurality of droplets, a first set of droplets associated with cells that are homozygous at the locus;
identifying, using the scDNA-seq data and from among the plurality of droplets not in the first set of droplets, a second set of droplets associated with more than two alleles at the locus; and
identifying, using the scDNA-seq data and from among the plurality of droplets not in the first or second sets of droplets, a third set of droplets associated with cells that are heterozygous at the locus.
